(19) United States
(12) Patent Application Publication
(10) Pub. No.: US 2024/0216343 A1
(43) Pub. Date: Jul. 4, 2024

(54) ENDOTHELIAL IK AND SK CHANNEL ACTIVATION TO TREAT PULMONARY HYPERTENSION

(71) Applicant: UNIVERSITY OF VIRGINIA PATENT FOUNDATION, Charlottesville, VA (US)

(72) Inventor: Swapnil Sonkusare, Charlottesville, VA (US)

(21) Appl. No.: 18/403,572

(22) Filed: Jan. 3, 2024

Related U.S. Application Data
(60) Provisional application No. 63/478,398, filed on Jan. 4, 2023.

(51) Int. Cl. A61K 31/426; A61K 31/404; A61K 31/4184; A61P 9/12

(52) U.S. Cl. CPC A61K 31/426; A61K 31/404; A61K 31/4184; A61P 9/12

(57) ABSTRACT

Disclosed are methods for treating pulmonary hypertension by activating the endothelial intermediate potassium (IK) and endothelial small potassium (SK) channels in a subject in need thereof.

### CROSS-REFERENCE TO RELATED APPLICATIONS

This application claims priority to U.S. Provisional Application No. 63/478,398 filed on Jan. 4, 2023, the content of which is incorporated by reference in its entirety.

### STATEMENT REGARDING FEDERALLY SPONSORED RESEARCH

This invention was made with government support under HL146914, HL142808, and HL157407 awarded by the National Institutes of Health. The government has certain rights in the invention.

### BACKGROUND OF THE INVENTION

Pulmonary hypertension (PH) is a progressive and debilitating disease characterized by elevated pulmonary arterial pressure (PAP) and pulmonary vascular resistance (PVR). Findings from the Registry to Evaluate Early and Long-term Pulmonary Arterial Hypertension Disease (REVEAL) report that, on average, patients with PAH have a survival rate of 85% in the first year and 49% within seven years. A lack of proper diagnosis and treatment of PH results in progressive right heart failure with a median survival rate of 2.8 years. Loss of endothelial function in small, resistance-sized pulmonary arteries (PAs) is a contributor to the pathogenesis of PH.

Current treatment options circumvent endothelial dysfunction in PH by directly increasing the levels of NO or prostacyclin in the extracellular milieu. However, these treatment options have limited efficacy and are associated with off-target effects on other cell types. An alternative strategy involving activation of an endothelial mechanism to lower PAP can be associated with fewer side effects. Therefore, identifying an endothelial mechanism that is not impaired in PH and could be easily activated can prove vital for designing a treatment strategy that lowers PAP in an endothelium-dependent manner.

### SUMMARY

In an aspect, provided herein is a method for treating pulmonary hypertension (PH) in a subject in need thereof, the method comprising administering to the subject a therapeutically effective amount of an activator of at least one of an endothelial intermediate potassium (IK) channel and an endothelial small potassium (SK) channel. In embodiments, the activator activates both of the IK channel and the SK channel. In embodiments, the activator is administered as a chronic treatment.

In embodiments, the activator is administered over the course of at least 1 week. In embodiments, the activator is administered at least once per day for at least 7 days. In embodiments, the activator is administered at least twice per day for at least 7 days.

In embodiments, the activator is a compound. In embodiments, the activator comprises at least one of SKA-31, NS309, and NS4591 or a pharmaceutically acceptable salt thereof. In embodiments, the activator is SKA-31 or a pharmaceutically acceptable salt thereof. In embodiments, the activator is NS309 or a pharmaceutically acceptable salt thereof. In embodiments, the activator is NS4591 or a pharmaceutically acceptable salt thereof. In embodiments, the subject is a human.

### BRIEF DESCRIPTION OF THE DRAWINGS

FIGS. 1A-1H. Endothelial IK/SK channel activity is not impaired in PAs from a mouse model of PH. (A) Representative right ventricular systolic pressure (RVSP; mm Hg) traces (left) and averaged RVSP values (right) in normoxic (N) mice and mice exposed to chronic hypoxia (CH; 4 weeks; 10% O₂) and SU5416 (Su+CH; n=12; *** P<0.001; t-test). (B) Average Fulton Index values in N and Su+CH mice (n=12; *** P<0.001; t-test). (C) Images showing 4th-order PAs (left) and ECs isolated from PAs in a patch-clamp experiment (D) Left, Representative images showing immunostaining for the EC marker CD31 and nuclear staining with DAPI in freshly isolated ECs from small PAs. (E) Left, Representative traces showing IK/SK currents in freshly isolated ECs from N (left) and Su+CH (right) mice in the presence of IK/SK channel activator NS309 (1 μmol/L), followed by sequential addition of the IK channel inhibitor TRAM-34 (1 μmol/L) and SK channel inhibitor apamin (Apa; 300 nmol/L). Currents were recorded in the whole-cell configuration of the patch-clamp technique. (F) Current density (pA/pF) plot of IK/SK currents in freshly isolated ECs from PAs of N and Su+CH mice in the presence of NS309 (1 μmol/L), followed by sequential addition of TRAM-34 (1 μmol/L) and Apa (300 nmol/L; n=5-7; *** P<0.001 vs. N [+NS309]; *** P<0.001 vs. Su+CH [+NS309]; two-way ANOVA). (G) Left, Representative traces showing IK/SK currents in freshly isolated ECs from N (top) and Su+CH (bottom) mice in the presence of 3 μmol/L free cytosolic Ca²⁺, followed by sequential addition of the IK channel inhibitor TRAM-34 (1 μmol/L) and SK channel inhibitor apamin (Apa; 300 nmol/L). Currents were recorded in the whole-cell configuration of the patch-clamp technique. (H) Current density (pA/pF) plot of IK/SK currents in freshly isolated ECs from PAs of N and Su+CH mice in the 3 μmol/L free cytosolic Ca²⁺, followed by sequential addition of TRAM-34 (1 μmol/L) and Apa (300 nmol/L; n=5-7; ** P<0.01 vs. Basal; *** P<0.001 vs. Basal; two-way ANOVA).

FIGS. 2A-2F. IK/SK channel-mediated dilation of small PAs is intact in a mouse model of PH. (A) Fourth-order PAs (left) were pressurized to 15 mm Hg and pre-constricted with the thromboxane A2 receptor agonist U46619 (30 nmol/L). Pressure myography traces (center and right) of PAs in response to NS309 (0.3-3 μmol/L). (B) Averaged percent dilation of PAs in response to NS309 (0.3-3 μmol/L; n=5-6). (C) Percent dilation of PAs that developed myogenic tone to NS309 (1 μmol/L; n=4). (D) Pressure myography traces (left) and averaged percent constriction (right) to TRAM-34 (1 μmol/L)+Apa (300 nmol/L; n=5) in small PAs from N and Su+CH mice. (E) Pressure myography traces (left) and averaged percent constriction (right) to the NOS inhibitor L-NNA (100 μmol/L; n=5; *** P<0.01 vs. N; t-test). (F) DAF-FM fluorescence analysis of endothelial NO levels in en face preparations of PAs from N and Su+CH mice (n=5;*** P<0.001 vs. N; t-test).

FIGS. 3A-3B. Acute administration of an IK/SK channel activator reduces PAP in PH. (A) Left, Representative RVSP (mm Hg) traces in N and Su+CH mice before and after acute treatment with the IK/SK channel activator SKA-31 (30 mg·kg⁻¹; intraperitoneally; i.p.). Right, Average RVSP values in N and Su+CH mice before and after acute treatment with SKA-31 (30 mg·kg⁻¹; i.p.; n=8; ** P<0.01 vs. no SKA-31 [Su+CH]; paired t-test). (B) Left, PAP (mm Hg) traces in isolated perfused lungs from N and Su+CH mice before and after acute treatment with NS309 (1 μmol/L). Right, Average PAP (mm Hg) in isolated perfused lungs from N and Su+CH mice before and after acute treatment with NS309 (1 μmol/L; n=10; ** P<0.01 vs. no SKA-31 [Su+CH]; paired t-test).

FIGS. 4A-4C. SMCs in small PAs from N and Su+CH mice do not express functional IK/SK channels. (A) Representative images showing immunostaining for the SMC marker α-actin and nuclear staining with DAPI in freshly isolated SMCs from small PAs. (B) Representative traces showing ionic currents in freshly isolated SMCs from N (top) and Su+CH (bottom) mice in the presence of NS309 (1 μmol/L), followed by addition of TRAM-34 (1 μmol/L) and Apa (300 nmol/L). Currents were recorded in the whole-cell configuration of the patch-clamp technique. (C) Current density (pA/pF) at 0 mV in freshly isolated SMCs from N and Su+CH mice at basal level and in the presence of NS309 (1 μmol/L), followed by TRAM-34 (1 μmol/L) and Apa (300 nmol/L; n=4).

FIGS. 5A-5C. Chronic treatment with an IK/SK channel activator decreases RVSP and vascular remodeling in PH. (A) Representative RVSP traces (left; mm Hg) and averaged RVSP values (center) in N and Su+CH mice after chronic (7 days; twice a day) treatment with SKA-31 (30 mg· kg⁻¹; i.p.; n=4; * P<0.05 vs. no SKA-31 [Su+CH]; t-test) or vehicle (−SKA-31). Right, Average Fulton Index values in N and Su+CH mice in the absence or presence of chronic treatment with SKA-31 (30 mg·kg⁻¹; i.p.; n=4; *** P<0.001 vs. no SKA-31 [Su+CH]; t-test). (B) Left, Masson Trichrome staining for collagen I in 5-μm-thick lower lobe lung sections from N and Su+CH mice before and after treatment with SKA-31 (30 mg kg⁻¹; i.p.; 7 days; twice a day). Right, Averaged collagen I area in PAs from N and Su+CH mice before and after treatment with SKA-31. Data were normalized by dividing by arterial circumference (n=4-6; ** P<0.01 vs. no SKA-31 [Su+CH]; two-way ANOVA). (C) Left, Representative images showing immunostaining for the SMC marker a-actin and nuclear staining with DAPI in PAs from N and Su+CH mice before and after treatment with SKA-31 (30 mg·kg⁻¹; i.p.; 7 days; twice a day). Right, Averaged α-actin area in PAs from N and Su+CH mice before and after treatment with SKA-31. Data were normalized by dividing by arterial circumference (PAs; n=5; *** P<0.001 vs. no SKA-31 [Su+CH]; two-way ANOVA).

FIG. 6. A pressure myography trace showing the diameter of a pulmonary artery from a human subject with PH (left). Pulmonary arteries were pre-constricted with U46619 (U466, thromboxane A2 receptor agonist). NS309 (IK/SK channel agonist) dilated the pulmonary arteries. Pulmonary artery diameter in the physiological solution with 0 mM extracellular Ca2+(0 Ca2+PSS) indicates the maximum passive diameter of the artery. Dilation of pulmonary arteries (right) in response to vehicle or NS309 treatment was calculated as a percentage of maximum passive diameter (n=3 arteries, unpaired t-test).

### DETAILED DESCRIPTION

Endothelium-dependent vasodilation of small pulmonary arteries (PAs) lowers the resting pulmonary arterial pressure (PAP). Moreover, a loss of endothelium-dependent vasodilation is a major contributor to elevated PAP in pulmonary hypertension (PH). Therefore, therapeutic strategies specifically targeting endothelial vasodilatory mechanisms can have beneficial effects on PAP in PH. In this regard, endothelial Ca²⁺ signaling mechanisms promote PA dilation and are therapeutic targets for lowering PAP in PH. Under homeostatic conditions, Ca²⁺ signals activate endothelial nitric oxide synthase (eNOS) and Ca²⁺-sensitive intermediate and small conductance potassium (IK and SK) channels to dilate PAs. eNOS activity and NO signaling are impaired in PH. However, the activity of IK/SK channels and its effect on PAP in PH have not previously been investigated. It is demonstrated herein that IK/SK channels can be targeted for lowering PAP in PH.

A mouse model of PH (chronic hypoxia+Sugen 5416, hereinafter CH+Su, for 3 weeks) showed elevated right ventricular systolic pressure (RVSP, an indirect indicator of PAP) and development of pulmonary arterial lesions. It was shown that adenosine triphosphate (ATP) dilates PAs through Ca²⁺-dependent activation of eNOS in normal mice (Daneva et al., PNAS, 2021). PA pressure myography experiments demonstrated that ATP was unable to dilate PAs from CH+Su mice, confirming a loss of eNOS-dependent vasodilation in these mice. However, PA dilation in response to IK/SK channel agonist NS309 (1 μM) was not different between PAs from control and CH+Su mice. Whole-cell patch-clamp studies in freshly isolated endothelial cells showed that IK/SK channel activity is not impaired in the endothelium from CH+Su mice. Finally, intraperitoneal administration of the IK/SK channel activator better suited for in vivo treatment, SKA-31 (30 mg/kg), lowered the RVSP in CH+Su mice. Cardiac functional magnetic resonance imaging studies revealed that acute SKA-31 administration does not alter the heart rate, stroke volume, ejection fraction, and cardiac output. The RVSP-lowering effect of SKA-31 was not observed in normal mice. Collectively, the data reveal unaltered IK/SK channel activity in PH and provide proof of principle that IK/SK activation can be used as a strategy for lowering PAP in PH.

Accordingly, provided herein is a method for treating pulmonary hypertension (PH) in a subject in need thereof, the method comprising administering to the subject a therapeutically effective amount of an activator of at least one of an endothelial intermediate potassium (IK) channel and an endothelial small potassium (SK) channel.

The terms “treating” and “to treat” PH includes the reducing, repressing, delaying or preventing PH. Treating PH may also include dilating pulmonary arteries, reducing/decreasing/lowering pulmonary arterial pressure (PAP), and reducing cardiac/vascular remodeling, reducing right ventricular systolic pressure (RVSP) and or right ventricular hypertrophy.

The term “subject” may be used interchangeably with the terms “individual” and “patient” and includes human and non-human mammalian subjects. As used herein, “a subject in need thereof” refers to a subject having or at risk of developing pulmonary hypertension or symptoms thereof.

An “activator” of an IK/SK channel is an agent that increases potassium ionic current through the IK/SK channel. In embodiments, the activator activates the IK channel. In other embodiments, the activator activates the SK channel. In preferred embodiments, the activator activates both the IK and SK channels.

The activator may be a compound. The activator may comprise at least one of SKA-31, NS309, and NS4591, or a pharmaceutically acceptable salt thereof. The term “pharmaceutically acceptable salt” as used herein, refers to salts of the compounds, which are substantially non-toxic to living organisms. Typical pharmaceutically acceptable salts include those salts prepared by reaction of the compounds as disclosed herein with a pharmaceutically acceptable mineral or organic acid or an organic or inorganic base. Such salts are known as acid addition and base addition salts. It will be appreciated by the skilled reader that most or all of the compounds as disclosed herein are capable of forming salts and that the salt forms of pharmaceuticals are commonly used, often because they are more readily crystallized and purified than are the free acids or bases.

Acids commonly employed to form acid addition salts may include inorganic acids such as hydrochloric acid, hydrobromic acid, hydroiodic acid, sulfuric acid, phosphoric acid, and the like, and organic acids such as p-toluenesulfonic, methanesulfonic acid, oxalic acid, p-bromophenylsulfonic acid, carbonic acid, succinic acid, citric acid, benzoic acid, acetic acid, and the like. Examples of suitable pharmaceutically acceptable salts may include the sulfate, pyrosulfate, bisulfate, sulfite, bisulfate, phosphate, monohydrogenphosphate, dihydrogenphosphate, metaphosphate, pyrophosphate, bromide, iodide, acetate, propionate, decanoate, caprylate, acrylate, formate, hydrochloride, dihydrochloride, isobutyrate, caproate, heptanoate, propiolate, oxalate, malonate, succinate, suberate, sebacate, fumarate, maleat-, butyne-1,4-dioate, hexyne-1,6-dioate, benzoate, chlorobenzoate, methylbenzoate, hydroxybenzoate, methoxybenzoate, phthalate, xylenesulfonate, phenylacetate, phenylpropionate, phenylbutyrate, citrate, lactate, a-hydroxybutyrate, glycolate, tartrate, methanesulfonate, propanesulfonate, naphthalene-1-sulfonate, naphthalene-2-sulfonate, mandelate, and the like.

Base addition salts include those derived from inorganic bases, such as ammonium or alkali or alkaline earth metal hydroxides, carbonates, bicarbonates, and the like. Bases useful in preparing such salts include sodium hydroxide, potassium hydroxide, ammonium hydroxide, potassium carbonate, sodium carbonate, sodium bicarbonate, potassium bicarbonate, calcium hydroxide, calcium carbonate, and the like.

The particular counter-ion forming a part of any salt of a compound disclosed herein may not be critical to the activity of the compound, so long as the salt as a whole is pharmacologically acceptable and as long as the counter-ion does not contribute undesired qualities to the salt as a whole. Undesired qualities may include undesirably solubility or toxicity.

Pharmaceutically acceptable esters and amides of the compounds can also be employed in the compositions and methods disclosed herein. Examples of suitable esters include alkyl, aryl, and aralkyl esters, such as methyl esters, ethyl esters, propyl esters, dodecyl esters, benzyl esters, and the like. Examples of suitable amides include unsubstituted amides, monosubstituted amides, and disubstituted amides, such as methyl amide, dimethyl amide, methyl ethyl amide, and the like.

In addition, the methods disclosed herein may be practiced using solvate forms of the compounds or salts, esters, and/or amides, thereof. Solvate forms may include ethanol solvates, hydrates, and the like.

The IK/SK channel activator may be prepared as a formulation or pharmaceutical composition. Inert ingredients and manner of formulation of the pharmaceutical compositions are conventional. The pharmaceutical compositions may be formulated according to conventional pharmaceutical practice (see, e.g., Remington: The Science and Practice of Pharmacy, 20th edition, 2000, ed. A. R. Gennaro, Lippincott Williams & Wilkins, Philadelphia, and Encyclopedia of Pharmaceutical Technology, eds. J. Swarbrick and J. C. Boylan, 1988-1999, Marcel Dekker, New York). The pharmaceutical compositions may be designed or intended for oral, rectal, nasal, systemic, topical or transmucosal (including buccal, sublingual, ocular, vaginal and rectal) and parenteral (including subcutaneous, intramuscular, intravenous, intraarterial, intradermal, intraperitoneal, intrathecal, intraocular and epidural) administration. Aqueous and non-aqueous liquid or cream formulations may be delivered by a parenteral, oral or topical route. The compositions may be present as an aqueous or a non-aqueous liquid formulation or a solid formulation suitable for administration by any route, e.g., oral, topical, buccal, sublingual, parenteral, aerosol, a depot such as a subcutaneous depot or an intraperitoneal or intramuscular depot. The pharmaceutical compositions may be lyophilized. The pharmaceutical compositions may contain auxiliary substances such as wetting or emulsifying agents, pH buffering agents, gelling or viscosity enhancing additives, preservatives, flavoring agents, colors, and the like, depending upon the route of administration and the preparation desired. For intravenous administration, suitable carriers include physiological saline, bacteriostatic water, Cremophor EL® (BASF, Parsippany, N.J., USA) or phosphate buffered saline (PBS). In all cases, a composition for parenteral administration must be sterile and should be formulated for ease of injectability. The composition should be stable under the conditions of manufacture and storage, and must be shielded from contamination by microorganisms such as bacteria and fungi.

The composition may further comprise a pharmaceutically acceptable carrier. The term “pharmaceutically acceptable carrier,” as used herein, means a non-toxic, inert solid, semi-solid or liquid filler, diluent, encapsulating material or formulation auxiliary of any type. Suitable pharmaceutically acceptable carriers include, but are not limited to, diluents, preservatives, solubilizers, emulsifiers, liposomes, nanoparticles and adjuvants. Some examples of materials which can serve as pharmaceutically acceptable carriers are sugars such as, but not limited to, lactose, glucose and sucrose; starches such as, but not limited to, corn starch and potato starch; cellulose and its derivatives such as, but not limited to, sodium carboxymethyl cellulose, ethyl cellulose and cellulose acetate; powdered tragacanth; malt; gelatin; talc; excipients such as, but not limited to, cocoa butter and suppository waxes; oils such as, but not limited to, peanut oil, cottonseed oil, safflower oil, sesame oil, olive oil, corn oil and soybean oil; glycols; such as propylene glycol; esters such as, but not limited to, ethyl oleate and ethyl laurate; agar; buffering agents such as, but not limited to, magnesium hydroxide and aluminum hydroxide; alginic acid; pyrogen-free water; isotonic saline; Ringer's solution; ethyl alcohol, and phosphate buffer solutions, as well as other non-toxic compatible lubricants such as, but not limited to, sodium lauryl sulfate and magnesium stearate, as well as coloring agents, releasing agents, coating agents, sweetening, flavoring and perfuming agents, preservatives and antioxidants can also be present in the composition, according to the judgment of the formulator.

Pharmaceutically acceptable carriers are well known to those skilled in the art and include, but are not limited to, 0.01 to 0.1 M and preferably 0.05M phosphate buffer or 0.9% saline. Additionally, such pharmaceutically acceptable carriers may be aqueous or non-aqueous solutions, suspensions, and emulsions. Examples of nonaqueous solvents are propylene glycol, polyethylene glycol, vegetable oils such as olive oil, and injectable organic esters such as ethyl oleate. Aqueous carriers include isotonic solutions, alcoholic/aqueous solutions, emulsions or suspensions, including saline and buffered media. A tabulation of ingredients listed by the above categories, may be found in the U.S. Pharmacopeia National Formulary, 1857-1859, (1990).

Some examples of the materials which can serve as pharmaceutically acceptable carriers are sugars, such as lactose, glucose and sucrose; starches such as corn starch and potato starch; cellulose and its derivatives such as sodium carboxymethyl cellulose, ethyl cellulose and cellulose acetate; powdered tragacanth; malt; gelatin; talc; excipients such as cocoa butter and suppository waxes; oils such as peanut oil, cottonseed oil, safflower oil, sesame oil, olive oil, corn oil and soybean oil; glycols, such as propylene glycol; polyols such as glycerin, sorbitol, mannitol and polyethylene glycol; esters such as ethyl oleate and ethyl laurate; agar; buffering agents such as magnesium hydroxide and aluminum hydroxide; alginic acid; pyrogen free water; isotonic saline; Ringer's solution, ethyl alcohol and phosphate buffer solutions, as well as other nontoxic compatible substances used in pharmaceutical formulations. Wetting agents, emulsifiers and lubricants such as sodium lauryl sulfate and magnesium stearate, as well as coloring agents, release agents, coating agents, sweetening, flavoring and perfuming agents, preservatives and antioxidants can also be present in the compositions, according to the desires of the formulator.

Examples of pharmaceutically acceptable antioxidants include water soluble antioxidants such as ascorbic acid, cysteine hydrochloride, sodium bisulfite, sodium metabisulfite, sodium sulfite and the like; oil-soluble antioxidants such as ascorbyl palmitate, butylated hydroxyanisole (BHA), butylated hydroxytoluene (BHT), lecithin, propyl gallate, alpha-tocopherol and the like; and metal-chelating agents such as citric acid, ethylenediamine tetraacetic acid (EDTA), sorbitol, tartaric acid, phosphoric acid and the like.

The composition may additionally include a biologically acceptable buffer to maintain a pH close to neutral (7.0-7.3). Such buffers preferably used are typically phosphates, carboxylates, and bicarbonates. More preferred buffering agents are sodium phosphate, potassium phosphate, sodium citrate, calcium lactate, sodium succinate, sodium glutamate, sodium bicarbonate, and potassium bicarbonate. The buffer may comprise about 0.0001-5% (w/v) of the vaccine formulation, more preferably about 0.001-1% (w/v). Other excipients, if desired, may be included as part of the final composition. The terms “about” and “approximately” shall generally mean an acceptable degree of error for the quantity measured given the nature or precision of the measurements. Typical, exemplary degrees of error are within 10%, and preferably within 5% of a given value or range of values. Alternatively, and particularly in biological systems, the terms “about” and “approximately” may mean values that are within an order of magnitude, preferably within 5-fold and more preferably within 2-fold of a given value. Numerical quantities given herein are approximate unless stated otherwise, meaning that the term “about” or “approximately” can be inferred when not expressly stated.

Solutions or suspensions used for parenteral, intradermal, or subcutaneous application can include the following components: a sterile diluent such as water for injection, saline solution, fixed oils, polyethylene glycols, glycerin, propylene glycol or other synthetic solvents; antibacterial agents such as benzyl alcohol or methyl parabens; antioxidants such as ascorbic acid or sodium bisulfite; chelating agents such as ethylenediaminetetraacetic acid (EDTA); buffers such as acetates, citrates or phosphates, and agents for the adjustment of tonicity, such as sodium chloride or dextrose. The pH can be adjusted with acids or bases, such as hydrochloric acid or sodium hydroxide. The preparation can be enclosed in ampoules, disposable syringes or multiple-dose vials made of glass or plastic. For convenience of the patient or treating physician, the dosing formulation can be provided in a kit containing all necessary equipment (e.g., vials of drug, vials of diluent, syringes and needles) for a course of treatment (e.g., 7 days of treatment).

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle, which contains a basic dispersion medium and the required other ingredients from those enumerated above. In the case of sterile powders for the preparation of sterile injectable solutions, typical methods of preparation include vacuum drying and freeze drying, which can yield a powder of the active ingredient plus any additional desired ingredient from a previously sterile-filtered solution thereof.

Capsules are prepared by mixing the compound with a suitable diluent and filling the proper amount of the mixture in capsules. The usual diluents include inert powdered substances (such as starches), powdered cellulose (especially crystalline and microcrystalline cellulose), sugars (such as fructose, mannitol and sucrose), grain flours, and similar edible powders. Tablets are prepared by direct compression, by wet granulation, or by dry granulation. Their formulations usually incorporate diluents, binders, lubricants, and disintegrators (in addition to the compounds). Typical diluents include, for example, various types of starch, lactose, mannitol, kaolin, calcium phosphate or sulfate, inorganic salts (such as sodium chloride), and powdered sugar. Powdered cellulose derivatives can also be used. Typical tablet binders include substances such as starch, gelatin, and sugars (e.g., lactose, fructose, glucose, and the like). Natural and synthetic gums can also be used, including acacia, alginates, methylcellulose, polyvinylpyrrolidine, and the like. Polyethylene glycol, ethylcellulose, and waxes can also serve as binders.

Tablets can be coated with sugar, e.g., as a flavor enhancer and sealant. The compounds also may be formulated as chewable tablets, by using large amounts of pleasant-tasting substances, such as mannitol, in the formulation. Instantly dissolving tablet-like formulations can also be employed, for example, to assure that the patient consumes the dosage form and to avoid the difficulty that some patients experience in swallowing solid objects. A lubricant can be used in a tablet formulation to prevent the tablet and punches from sticking in the die. The lubricant can be chosen from such slippery solids as talc, magnesium and calcium stearate, stearic acid, and hydrogenated vegetable oils. Tablets can also contain disintegrators. Disintegrators are substances that swell when wetted to break up the tablet and release the compound. They include starches, clays, celluloses, algins, and gums. As further illustration, corn and potato starches, methylcellulose, agar, bentonite, wood cellulose, powdered natural sponge, cation-exchange resins, alginic acid, guar gum, citrus pulp, sodium lauryl sulfate, and carboxymethylcellulose can be used.

Compositions can be formulated as enteric formulations, for example, to protect the active ingredient from the strongly acid contents of the stomach. Such formulations can be created by coating a solid dosage form with a film of a polymer which is insoluble in acid environments and soluble in basic environments. Illustrative films include cellulose acetate phthalate, polyvinyl acetate phthalate, hydroxypropyl methylcellulose phthalate, and hydroxypropyl methylcellulose acetate succinate.

Transdermal patches can also be used to deliver the compositions. Transdermal patches can include a resinous composition in which a compound will dissolve or partially dissolve; and a film which protects the composition, and which holds the resinous composition in contact with the skin. Other, more complicated patch compositions can also be used, such as those having a membrane pierced with a plurality of pores through which the drugs are pumped by osmotic action.

As one skilled in the art will also appreciate, the formulation can be prepared with materials (e.g., actives excipients, carriers (such as cyclodextrins), diluents, etc.) having properties (e.g., purity) that render the formulation suitable for administration to humans. Alternatively, the formulation can be prepared with materials having purity and/or other properties that render the formulation suitable for administration to non-human subjects, but not suitable for administration to humans.

As used herein, the term “administering”, refers to dispensing, delivering, or applying the activator of the IK/SK channels, to a subject by any suitable route for delivery of the activator to the desired location in the subject, including delivery by either the parenteral or oral route, intramuscular injection, subcutaneous/intradermal injection, intravenous injection, intraperitoneal injection, intrathecal administration, buccal administration, transdermal delivery, topical administration, and administration by the intranasal or respiratory tract route.

The terms “effective amount” or “therapeutically effective amount” refer to an amount sufficient to effect beneficial or desirable biological and/or clinical results. The amount of the agent or pharmaceutical composition that is therapeutically effective may vary depending on the particular condition of the subject. Appropriate dosages may be determined, for example, by extrapolation from cell culture assays, animal studies, or human clinical trials taking into account body weight of the patient, absorption rate, half-life, disease severity and the like. The dosage lies preferably within a range of circulating concentrations that include the ED50 with little or no toxicity. The dosage may vary within this range depending upon the dosage form employed and the route of administration utilized. A dose can be formulated in animal models to achieve a circulating plasma concentration range that includes the IC50 (i.e., the concentration of the test compound which achieves a half-maximal inhibition of symptoms) as determined in cell culture. Such information can be used to more accurately determine useful doses in humans. Levels in plasma may be measured, for example, by high performance liquid chromatography.

The IK/SK channel activator may be administered as a chronic treatment, meaning a treatment longer than one day or one dose. The activator may be administered over the course of at least one week. The activator may be administered at least one per day for at least seven days. The activator may be administered at least twice per day for at least seven days. The activator may be administered as a daily dosage for an unlimited number of days.

A typical daily dose may contain from about 0.01 mg/kg to about 100 mg/kg (such as from about 0.05 mg/kg to about 50 mg/kg and/or from about 0.1 mg/kg to about 25 mg/kg) of each compound used in the present method of treatment.

Compositions can be formulated in a unit dosage form, each dosage containing from about 1 to about 500 mg of each compound individually or in a single unit dosage form, such as from about 5 to about 300 mg, from about 10 to about 100 mg, and/or about 25 mg. The term “unit dosage form” refers to a physically discrete unit suitable as unitary dosages for a patient, each unit containing a predetermined quantity of active material calculated to produce the desired therapeutic effect, in association with a suitable pharmaceutical carrier, diluent, or excipient.

### Miscellaneous

References in the specification to “one embodiment,” “an embodiment,” etc., indicate that the embodiment described may include a particular aspect, feature, structure, moiety, or characteristic, but not every embodiment necessarily includes that aspect, feature, structure, moiety, or characteristic. Moreover, such phrases may, but do not necessarily, refer to the same embodiment referred to in other portions of the specification. Further, when a particular aspect, feature, structure, moiety, or characteristic is described in connection with an embodiment, it is within the knowledge of one skilled in the art to affect or connect such aspect, feature, structure, moiety, or characteristic with other embodiments, whether or not explicitly described.

The singular forms “a,” “an,” and “the” include plural reference unless the context clearly dictates otherwise. Thus, for example, a reference to “a compound” includes a plurality of such compounds, so that a compound X includes a plurality of compounds X. It is further noted that the claims may be drafted to exclude any optional element. As such, this statement is intended to serve as antecedent basis for the use of exclusive terminology, such as “solely,” “only,” and the like, in connection with any element described herein, and/or the recitation of claim elements or use of “negative” limitations.

The term “and/or” means any one of the items, any combination of the items, or all of the items with which this term is associated. The phrase “one or more” is readily understood by one of skill in the art, particularly when read in context of its usage. For example, one or more substituents on a phenyl ring refers to one to five, or one to four, for example if the phenyl ring is disubstituted.

As used herein, “or” should be understood to have the same meaning as “and/or” as defined above. For example, when separating a listing of items, “and/or” or “or” shall be interpreted as being inclusive, e.g., the inclusion of at least one, but also including more than one, of a number of items, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e., “one or the other but not both”) when preceded by terms of exclusivity, such as “either,” “one of,” “only one of,” or “exactly one of.”

As used herein, the terms “including,” “includes,” “having,” “has,” “with,” or variants thereof, are intended to be inclusive similar to the term “comprising.”

The term “about” can refer to a variation of +5%, +10%, +20%, or +25% of the value specified. For example, “about 50” percent can in some embodiments carry a variation from 45 to 55 percent. For integer ranges, the term “about” can include one or two integers greater than and/or less than a recited integer at each end of the range. Unless indicated otherwise herein, the term “about” is intended to include values, e.g., weight percentages, proximate to the recited range that are equivalent in terms of the functionality of the individual ingredient, the composition, or the embodiment. The term about can also modify the endpoints of a recited range as discuss above in this paragraph.

As will be understood by the skilled artisan, all numbers, including those expressing quantities of ingredients, properties such as molecular weight, reaction conditions, and so forth, are approximations and are understood as being optionally modified in all instances by the term “about.” These values can vary depending upon the desired properties sought to be obtained by those skilled in the art utilizing the teachings of the descriptions herein. It is also understood that such values inherently contain variability necessarily resulting from the standard deviations found in their respective testing measurements.

As will be understood by one skilled in the art, for any and all purposes, particularly in terms of providing a written description, all ranges recited herein also encompass any and all possible sub-ranges and combinations of sub-ranges thereof, as well as the individual values making up the range, particularly integer values. A recited range (e.g., weight percentages or carbon groups) includes each specific value, integer, decimal, or identity within the range. Any listed range can be easily recognized as sufficiently describing and enabling the same range being broken down into at least equal halves, thirds, quarters, fifths, or tenths. As a non-limiting example, each range discussed herein can be readily broken down into a lower third, middle third and upper third, etc. As will also be understood by one skilled in the art, all language such as “up to,” “at least,” “greater than,” “less than,” “more than,” “or more,” and the like, include the number recited and such terms refer to ranges that can be subsequently broken down into sub-ranges as discussed above. In the same manner, all ratios recited herein also include all sub-ratios falling within the broader ratio. Accordingly, specific values recited for radicals, substituents, and ranges, are for illustration only; they do not exclude other defined values or other values within defined ranges for radicals and substituents.

One skilled in the art will also readily recognize that where members are grouped together in a common manner, such as in a Markush group, the invention encompasses not only the entire group listed as a whole, but each member of the group individually and all possible subgroups of the main group.

Additionally, for all purposes, the invention encompasses not only the main group, but also the main group absent one or more of the group members. The invention therefore envisages the explicit exclusion of any one or more of members of a recited group. Accordingly, provisos may apply to any of the disclosed categories or embodiments whereby any one or more of the recited elements, species, or embodiments, may be excluded from such categories or embodiments, for example, for use in an explicit negative limitation.

The term “contacting” refers to the act of touching, making contact, or of bringing to immediate or close proximity, including at the cellular or molecular level, for example, to bring about a physiological reaction, a chemical reaction, or a physical change, e.g., in a solution, in a reaction mixture, in vitro, or in vivo.

The term “standard,” as used herein, refers to something used for comparison. For example, it can be a known standard agent or compound which is administered and used for comparing results when administering a test compound, or it can be a standard parameter or function which is measured to obtain a control value when measuring an effect of an agent or compound on a parameter or function. Standard can also refer to an “internal standard”, such as an agent or compound which is added at known amounts to a sample and is useful in determining such things as purification or recovery rates when a sample is processed or subjected to purification or extraction procedures before a marker of interest is measured. Internal standards are often a purified marker of interest which has been labeled, such as with a radioactive isotope, allowing it to be distinguished from an endogenous marker.

Methods involving conventional molecular biology techniques are described herein. Such techniques are generally known in the art and are described in detail in methodology treatises, such as Molecular Cloning: A Laboratory Manual, 2nd ed., vol. 1-3, ed. Sambrook et al., Cold Spring Harbor Laboratory Press, Cold Spring Harbor, N.Y., 1989; and Current Protocols in Molecular Biology, ed. Ausubel et al., Greene Publishing and Wiley-Interscience, New York, 1992 (with periodic updates). Methods for chemical synthesis of nucleic acids are discussed, for example, in Beaucage and Carruthers, Tetra. Letts. 22: 1859-1862, 1981, and Matteucci et al., J. Am. Chem. Soc. 103:3185, 1981.

The definitions provided herein are included to provide a clear and consistent understanding of the specification and claims. All other terms and phrases used in this specification have their ordinary meanings as one of skill in the art would understand. Such ordinary meanings may be obtained by reference to technical dictionaries, such as Hawley's Condensed Chemical Dictionary 14th Edition, by R. J. Lewis, John Wiley & Sons, New York, N.Y., 2001.

All publications, patents, and patent applications, Genbank sequences, websites and other published materials referred to throughout the disclosure herein are herein incorporated by reference to the same extent as if each individual publication, patent, or patent application, Genbank sequences, websites and other published materials was specifically and individually indicated to be incorporated by reference. In the event that the definition of a term incorporated by reference conflicts with a term defined herein, this specification shall control.

### EXAMPLES

### Example 1

Endothelial intermediate (IK)- and small (SK)-conductance, Ca²⁺-activated K channels remain largely unexplored as therapeutic targets in PH. Studies in systemic arteries indicate a role for IK and SK channels in endothelium-dependent vasodilation (9) and blood pressure regulation (10). Increases in endothelial Ca²⁺ activate IK and SK channels, hyperpolarizing EC and nearby SMC membranes and causing vasodilation. Moreover, the activity of endothelial IK/SK channels is reduced in cardiovascular disorders, including hypertension, obesity, and diabetes (11-16). It was previously reported that functional IK and SK channels are present in ECs from small PAs and that IK/SK channel activation dilates PAs (10). However, direct recordings of endothelial IK/SK channel activity in PH are not available in the literature. Assessing the activity of endothelial IK/SK channels in PH is a step in determining whether these channels can be targeted to lower PAP in PH.

NO is the predominant vasodilator molecule in small PAs (17, 18). Under normal conditions, an increase in endothelial Ca²⁺ activates endothelial nitric oxide synthase (eNOS). Endothelium-derived NO then diffuses to SMCs, increasing cyclic guanosine monophosphate (cGMP) levels and cGMP-dependent kinase (PKG) activity to cause SMC relaxation (19). Endothelial Ca²⁺ elevation, eNOS activity, and NO-cGMP signaling are impaired in PAs from mouse models of PH and PH patients (6, 18). Moreover, the levels of prostacyclin, another endothelium-derived vasodilator, are also reduced in the lungs of PH patients (7). Although IK/SK channels are needed for endothelium-dependent dilation of systemic arteries, whether they can be targeted to lower PAP in PH is not known.

Herein, it is shown that endothelial IK/SK channel activation lowers PAP in PH utilizing a SU5416 plus chronic hypoxia (Su+CH) mouse model of PH. Su+CH mice showed elevated PAP and pulmonary arterial lesions. Acute treatment with the IK/SK channel agonist SKA-31 lowered PAP and right ventricular systolic pressure (RVSP) in Su+CH mice but not in control mice. Moreover, chronic treatment with SKA-31 reduced pulmonary artery lesions and right ventricular hypertrophy in Su+CH mice. Notably, the activity of IK and SK channels was not impaired in PH, and IK/SK channel-induced vasodilation of small PAs was also unaffected. Collectively, the data demonstrate that the endothelial IK/SK channel pathway for vasodilation is not altered in PH and provide proof-of-principle that IK/SK channel activation is a therapeutic strategy for lowering PAP and PA remodeling in PH.

### Materials and Methods

### Drugs

NS309 (C8H4C12N202), TRAM-34 and apamin were purchased from Tocris Bioscience (Minneapolis; MN; USA). U46619 was purchased from Cayman Chemicals (Ann Arbor; MI; USA). SU5416, SKA-31 (C11H8N2S; naphtho[1,2-d]thiazol-2-ylamine), and sodium nitroprusside (SNP) were purchased from Sigma Aldrich (St. Louis; MO; USA).

### Animal Protocols

All animal studies were approved by the University of Virginia Animal Care and Use Committee. Male C57BL6/J mice (10-14 weeks old; The Jackson Laboratory; Bar Harbor; ME;

USA) were used for this study. Mice were housed in an enriched environment and maintained under a 12:12 h light/dark photocycle at ˜23° ° C. with fresh tap water and a standard chow diet available ad libitum. Mice were sacrificed with pentobarbital (90 mg·kg⁻¹; i.p.; UVA Hospital Pharmacy; Charlottesville; VA; USA) followed by cranial dislocation for lung tissue harvesting.

Mouse model of PH

PH was induced by exposing C57BL6/J mice to chronic hypoxia (CH; 10% 02: 4 weeks) with concurrent treatment with the receptor tyrosine kinase inhibitor, SU5416 (20 mg·kg⁻¹), administered subcutaneously (s.c.) once a week (20). SU5416 was dissolved in DMSO/PEG 400 (50/50). Mice were exposed to CH conditions in a vinyl hypoxic chamber (Coy Laboratory Products; Inc. Grass Lake; MI; USA) connected to an auto purge airlock inlet. Oxygen concentration in the glove box was regulated by an oxygen controller and oxygen sensor (Coy Laboratory Products, Inc.). Control mice were maintained in room air for 4 weeks.

For PA remodeling studies, mice in the experimental group were treated with the IK/SK channel activator, SKA-31 (30 mg·kg⁻¹; i.p.), twice a day for the final 7 days of Su+CH exposure. Control mice were exposed to Su+CH for 4 weeks but were injected with vehicle instead of SKA-31. At the end of week 4 of Su+CH exposure, mice were euthanized with pentobarbital (90 mg·kg⁻¹; i.p.; UVA Hospital Pharmacy) and left lungs were perfused with the NO donor and fast-acting vasodilator, SNP (50 μmol/L; Sigma Aldrich), followed by incubation overnight at room temperature with 4% paraformaldehyde (PFA) for histology and immunostaining.

### Measurement of RVSP and Fulton Index

Mice were anesthetized with pentobarbital (50 mg·kg⁻¹; i.p.), and bupivacaine HCl (100 μL of 0.25% solution; s.c.) was used to numb the dissection site on the mouse. RVSP was measured as an indirect indicator of PAP (18, 21). Mice were cannulated with a Mikro-Tip pressure catheter (SPR-671; Millar Instruments; Houston; TX; USA), connected to a bridge amp (FE221) and a PowerLab 4/35 4-channel recorder (ADInstruments; Colorado Springs; CO; USA), through the external jugular vein into the right ventricle. Right ventricular pressure was acquired and analyzed using LabChart8 software (ADInstruments). In the subgroup of mice treated with SKA-31 (30 mg·kg⁻¹; i.p.), RVSP was measured before SKA-31 injection and 20 minutes after. A stable 3-minute recording was acquired for all animals, and a 1-minute continuous segment was used for data analysis. Where necessary, traces were digitally filtered using a low-pass filter at a cut-off frequency of 50 Hz. At the end of experiments, mice were euthanized, and their hearts were isolated for right ventricular hypertrophy analysis. Right ventricular hypertrophy was determined by calculating the Fulton index, a ratio of the right ventricular (RV) heart weight over the left ventricular (LV) plus septum (S) weight (RV/LV+S).

### Measurement of PAP

PAP was evaluated using an IPL-1 ex vivo murine lung perfusion system (Harvard Apparatus; Holliston; MA; USA) as previously described (22, 23). Briefly, mice were anesthetized with isoflurane, after which a tracheostomy was performed, and animals were ventilated with room air at 150 strokes/min and a stroke volume of 200 mL. Animals were exsanguinated by transecting the inferior vena cava. The main pulmonary artery was cannulated through the right ventricle, and the left ventricle was tube-vented through a small incision at the apex of the heart. The lungs were then perfused at a constant flow rate of 0.5 mL min 1 with Krebs-Henseleit buffer (11 mmol/L glucose; 1.2 mmol/L MgSO₄; 1.2 mmol/L KH₂PO₄; 4.7 mmol/L KCl; 118 mmol/L NaCl; 1.25 mmol/L CaCl₂), and 25 mmol/L NaCO₃). The perfusate buffer and isolated lungs were maintained at 37° C. using a circulating water bath. Once properly perfused and ventilated, the isolated lungs were maintained on the system for a 10-minute equilibration period, after which hemodynamic and pulmonary parameters were recorded using the PULMODYN data acquisition system (Hugo Sachs Elektronik; Breisgau; Germany).

### Pressure Myography

Mouse fourth-order PAs (˜50 μm) were cannulated on glass micropipettes in a pressure myography chamber (The Instrumentation and Model Facility; University of Vermont; Burlington; VT; USA) and pressurized to a physiological pressure of 15 mm Hg (24). Briefly, freshly dissected lungs were placed in ice cold HEPES buffer in a dissection plate. The left lung was stretched and pinned down, with ventral side facing up. First, pulmonary veins, and then the airway were cut open to gain access to the pulmonary arteries underneath. Once PAs were visible, they were gently separated from surrounding tissue (25). Arteries were superfused with bicarbonate-physiological salt solution (PSS; 119 mmol/L NaCl; 4.7 mmol/L KCl; 1.2 mmol/L KH₂PO₄; 1.2 mmol/L MgCl₂ hexahydrate; 2.5 mmol/L CaCl₂) dihydrate; 7 mmol/L dextrose; and 24 mmol/L NaHCO₃) at 37° ° C. and bubbled with 20% O₂/5% CO₂ to maintain the pH at 7.4. Approximately 20% PAs from normal mice develop myogenic tone, whereas ˜65% PA from Su+CH mice show myogenic tone at 15 mm Hg. Due to the relatively small percentage of PAs from normal mice showing myogenic tone, we pre-constricted all PAs with 30 nmol/L U46619 (a thromboxane A2 receptor agonist). More than 95% arteries showed a constriction in response to U46619 in both normal and PH groups. U46619 was added to the chamber immediately, before the arteries could develop myogenic tone. Before measuring vascular reactivity, arteries were treated with NS309 (1 μmol/L), a direct activator of endothelial IK/SK channels, to assess endothelial health. Arteries that failed to dilate completely after treatment with NS309 were discarded. Changes in arterial diameter were recorded at a 60-ms frame rate using a charge-coupled device camera and edge-detection software (IonOptix LLC; Westwood; MA; USA) (11, 26). At the end of each experiment, Ca²⁺-free PSS (119 mmol/L NaCl; 4.7 mmol/L KCl; 1.2 mmol/L KH₂PO₄; 1.2 mmol/L MgCl₂ hexahydrate; 7 mmol/L dextrose; 24 mmol/L NaHCO₃; and 5 mmol/L EGTA) was applied to assess the maximum passive diameter. Percent constriction was calculated by:

$$[ ( {Diameter}_{before} - {Diameter}_{after} ) / {Diameter}_{before} ] \times 100 , \qquad ( 1 )$$

where Diameter_{before} is the diameter of the artery before treatment and Diameter_{after} is the diameter after treatment. Percent dilation was calculated by:

$$[ \text{} ( {Diameter}_{dilated} - {Diameter}_{basal} ) \text{} / ( {Diameter}_{Ca - free} - {Diameter}_{basal} ] \times 100 , \qquad ( 2 )$$

where Diameter_{basal} is the stable diameter before drug treatment, Diameter_{dilated} is the diameter after drug treatment, and Diameter_{ca-free} is the maximum passive diameter.

### Immunostaining

Isolated left lungs were perfused with PBS containing 50 μmol/L SNP and fixed in 4% PFA overnight at room temperature before paraffin embedding and sectioning. After sectioning paraffinized lungs, sections (5-μm thick) were deparaffinized and incubated in endogenous peroxidase (30%) for 30 minutes at room temperature, followed by a 5-minute wash with double-distilled H₂O. For antigen retrieval, sections were microwaved in a citrate-based antigen unmasking solution (Vector Laboratories; Newark; CA; USA) for 20 minutes with brief pauses to avoid boiling. After allowing slides to cool in the antigen unmasking solution for 1 hour at room temperature, sections were permeabilized by incubating with 0.2% Triton-X for 30 minutes on a shaking rocker at room temperature, then blocked by incubating with 0.2% Triton-X, 5% normal donkey serum (ab7475; Abcam; Cambridge; MA; USA) and fish skin gelatin (FSG; Sigma Aldrich) for 1 hour. Thereafter, sections were incubated for 1 hour at room temperature with fluorescein isothiocyanate (FITC)-conjugated monoclonal anti-α-actin primary antibody (1:500; F3777; Sigma Aldrich) and washed first in PBS containing FSG for 5 minutes and then in PBS alone for 5 minutes (three times). After washing in PBS, nuclei were stained by incubating with 0.3 μmol/L 4′,6-diamidino-2-phenylindole (DAPI; Invitrogen; Carlsbad; CA; USA) for 10 minutes at room temperature. Images were acquired along the z-axis (optical slice thickness; 0.1 μm) using an Andor Dragonfly 505 confocal spinning-disk system (Oxford Instruments; Abingdon; UK) and a Leica DMi8 microscope with a 40X objective (NA 1.1; Leica Microsystems; Wetzlar; Germany). Images were extracted with IMARIS version 9.3 and analyzed with Image J. Data were normalized by dividing the collagen fiber area by the arterial diameter.

Isolated ECs and SMCs were fixed by incubation with 4% PFA at room temperature for 15 minutes and then permeabilized by treatment with PBS containing 0.2% Triton-X for 1 hour. The cells were then treated with 5% normal donkey serum for 1 hour. ECs were subsequently incubated with monoclonal CD31 antibody (1:100; #RM5201; Invitrogen (18)) for 1 hour at room temperature. SMCs were incubated with FITC-conjugated anti-α-actin antibody (1:500; F3777; Sigma Aldrich (18, 27) for 1 hour at room temperature. After washing the cells three times with PBS, nuclei were stained by incubating with 0.3 μmol/L DAPI (Invitrogen) for 10 minutes at room temperature in the dark.

Patch-Clamp Analysis of Freshly Isolated ECs and SMCs from Small PAs

ECs were freshly isolated from PAs following a previously established protocol (21, 24). Briefly, PAs were incubated in dissociation solution (55 mmol/L NaCl; 80 mmol/L Na glutamate; 6 mmol/L KCl; 2 mmol/L MgCl₂; 0.1 mmol/L CaCl₂); 10 mmol/L glucose; 10 mmol/L HEPES; pH 7.3) containing Worthington neutral protease (0.5 mg/mL; Worthington Biochemical Corporation; Lakewood; NJ; USA) for 30 minutes at 37° C. With this protocol for endothelial cell isolation, more than 95% of cells are CD31-positive.

For SMC isolation (27), arterial segments were transferred to a 12×75 mm borosilicate glass culture tube containing 1 mL dissociation solution (145 mmol/L NaCl; 4 mmol/L KCl; 1 mmol/L MgCl₂; 10 mmol/L HEPES; 0.05 mmol/L CaCl₂), 10 mmol/L glucose; pH 7.3) and 0.5 mg/mL bovine serum albumin (BSA) and incubated for 10 minutes at room temperature (˜24° C.). The solution was then replaced with 1 mL dissociation solution containing 1 mg/mL papain (Sigma Aldrich) and 0.5 mg/mL dithiothreitol (Sigma Aldrich), and incubation was continued at 37° C. for 20 minutes. Thereafter, 0.5 mL of the papain solution was carefully removed without displacing the arterial segments and replaced with 0.5 mL dissociation solution containing 2 mg/mL collagenase type IV (Worthington Biochemical Corporation), 0.5 mg/mL elastase (Sigma Aldrich) and 1 mg/mL soybean trypsin inhibitor (Sigma Aldrich). After incubating at 37° C. for 5 minutes, the enzyme solution was removed and replaced with cold dissociation solution containing BSA. The tube containing digested arteries was placed on ice, and the solution was gently triturated every 15 minutes for 1 hour to yield a single-cell suspension. SMCs were identified by their elongated shape and α-actin immunostaining.

Whole-cell currents were measured at room temperature using conventional whole-cell patch-clamp electrophysiology. The bath solution consisted of 10 mmol/L HEPES, 134 mmol/L NaCl, 6 mmol/L KCl, 2 mmol/L CaCl₂), 1 mmol/L MgCl₂ hexahydrate, and 7 mmol/L dextrose (adjusted to pH 7.4 with NaOH). The intracellular solution consisted of 10 mmol/L HEPES, 30 mmol/L KCl, 10 mmol/L NaCl, 110 mmol/L K-aspartate and 1 mmol/L MgCl₂ (adjusted to pH 7.2 with NaOH). The pipette solution for conventional patch clamp consisted of 10 mmol/L HEPES, 123.2 mmol/L KCl, 10 mmol/L NaCl, 5.5 mmol/L MgCl₂, 0.2 mmol/L CaCl₂) and 5 mmol/L HEDTA (adjusted to pH 7.2 with 16.8 mm KOH) and contained 3 μm free-Ca²⁺ and 1 mmol/L free-Mg²⁺, as calculated using the Max-Chelator program (Chris Patton; Stanford University; Stanford; CA; USA). The voltage-clamp protocol involved 250-ms voltage ramps from −100 mV to +50 mV from a holding potential of −50 mV. Patch electrodes were pulled from borosilicate glass (O.D.: 1.5 mm; I.D.: 1.17 mm; Sutter Instruments; Novato; CA; USA) using a Narishige PC-100 puller (Narishige International USA; Inc.; Amityville; NY; USA) and polished using a MicroForge MF-830 polisher (Narishige International USA). The pipette resistance was 3-5 mQ. IK and SK channel currents were elicited by adding 1 μmol/L NS309 (IK/SK channel activator) to the bath solution. IK/SK channel currents were inhibited by adding 1 μmol/L TRAM-34 (IK channel inhibitor) and 300 nmol/L Apamin (Apa; SK channel inhibitor) to the bath solution. The effect of each drug was studied 5 minutes after its addition. Data were acquired using a Multiclamp 700B amplifier connected to a Digidata 1550B system and analyzed using Clampfit 11.1 software (Molecular Devices, San Jose, CA, USA).

### Histology

Isolated left lungs were perfused with phosphate-buffered saline (PBS) containing 50 μmol/L SNP and fixed by incubating overnight at room temperature in 4% PFA. Tissue samples were then paraffin embedded, sectioning to a thickness of 5 μm, and stained with Masson's Trichrome (Polysciences, Inc.; Warrington; PA; USA). Collagen fiber thickness was calculated based on the area of collagen fiber staining (blue) using NIH Image J software. Data were normalized by dividing collagen fiber area by arterial diameter. Histology images were captured using a Leica DMIL LED microscope with a Leica DMC6200 camera and LAS X Software (Leica Microsystems Inc.; Buffalo Grove; IL; USA).

NO imaging with DAF-FM

NO was imaged by fluorescence microscopy using DAF-FM (4-amino-5 methylamino-2′,7′-difluorofluorescein diacetate), which forms a fluorescent triazole compound after binding NO. DAF-FM was dissolved in HEPES-PSS containing 0.02% pluronic acid (17) to obtain a solution with a final concentration of 5 μmol/L. Fourth-order PAs were pinned down en face on a Sylgard block and loaded with 5 μmol/L DAF-FM for 20 minutes at 30° C. in the dark. DAF-FM was excited at 488 nm, and emission was collected with a 525/36-nm band-pass filter. Images were captured across the z-axis (slice thickness; 0.1 μm) from the surface of the endothelium to the bottom where the EC layer encounters the SMC layer. Baseline NO release was studied in PAs from control (normoxic) and Su+CH mice. Custom-designed software written by Dr. Adrian Bonev (SparkAn) was used to analyze DAF-FM images (26) (https://github.com/vesselman/SparkAn). Arbitrary fluorescence intensity per cell was measured by manually drawing an outline around each EC, establishing the entire cell as a region of interest. Background fluorescence (intensity without laser) was subtracted from the arbitrary fluorescence value obtained for each cell, and the fluorescence values of each cell were averaged to obtain a single fluorescence number for the specific field.

### Statistics

Results are presented as means±standard error of the mean (SEM). Data were obtained from at least three mice in experiments performed on at least two independent groups. All data are presented graphically using CorelDraw ×9 (Corel Corp.; Ottawa; ON; Canada) and were analyzed statistically using OriginPro (version 7.5; OriginLab Corp.; Northampton; MA; USA), Prism (version 8; GraphPad Software Inc.; San Diego; CA; USA) and MATLAB R2019b (MathWorks; Natick; MA; USA). The normality of data was determined by performing a Shapiro-Wilk test. Data were analyzed using two-tailed, paired or independent t-tests for comparison of data collected from two different treatments, or one-way or two-way analysis of variance (ANOVA) for analysis of statistical differences among more than two different treatments. Statistical significance was determined as a P value less than 0.05; individual P-values (*P<0.05; ** P<0.01; *** P<0.001) are indicated in figure legends.

### Results

### Endothelial IK and SK Channel Activity in Small PAs is not Altered in a Mouse Model of PH

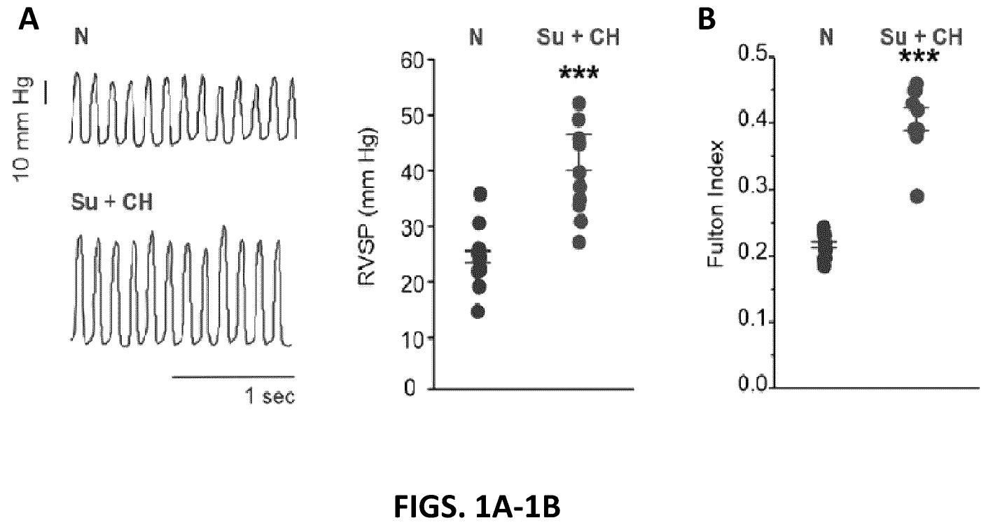

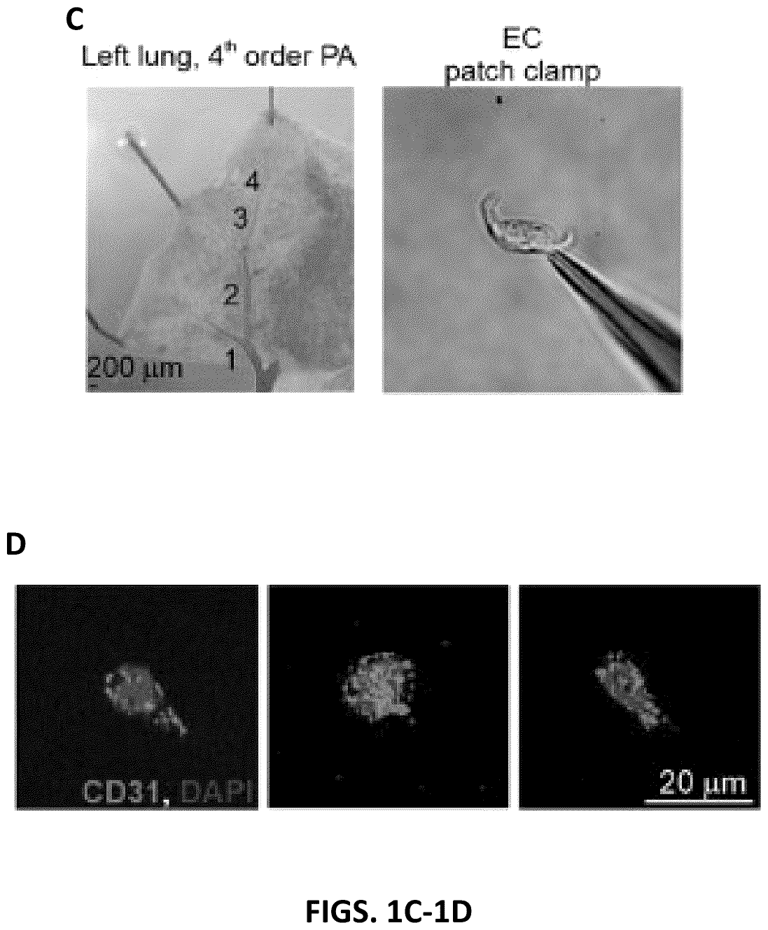

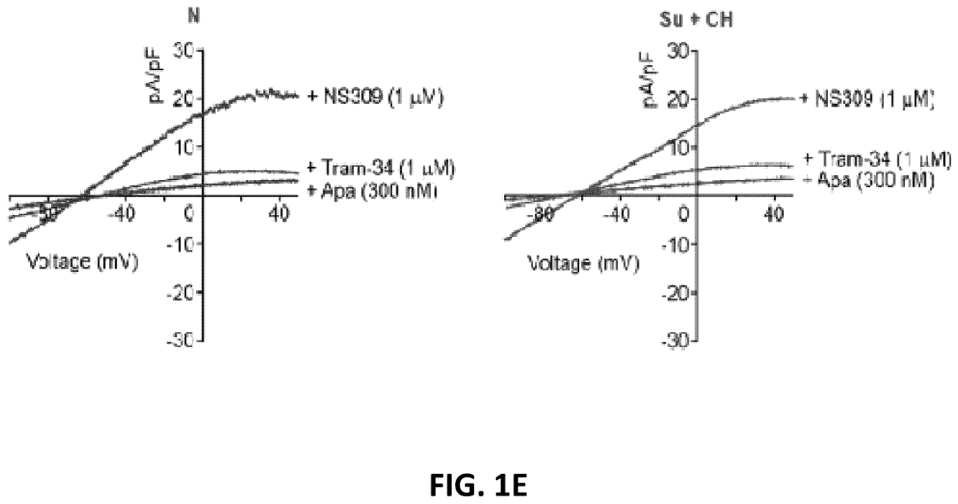

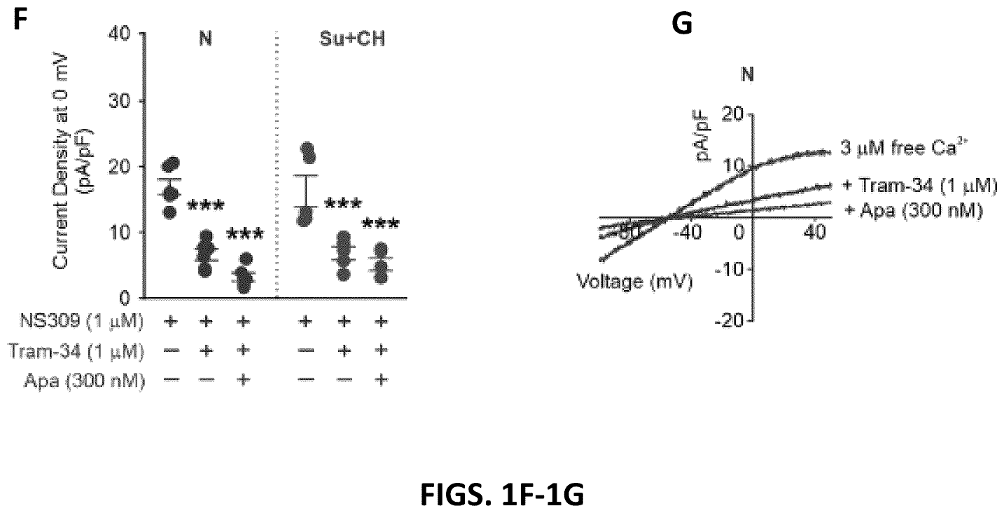

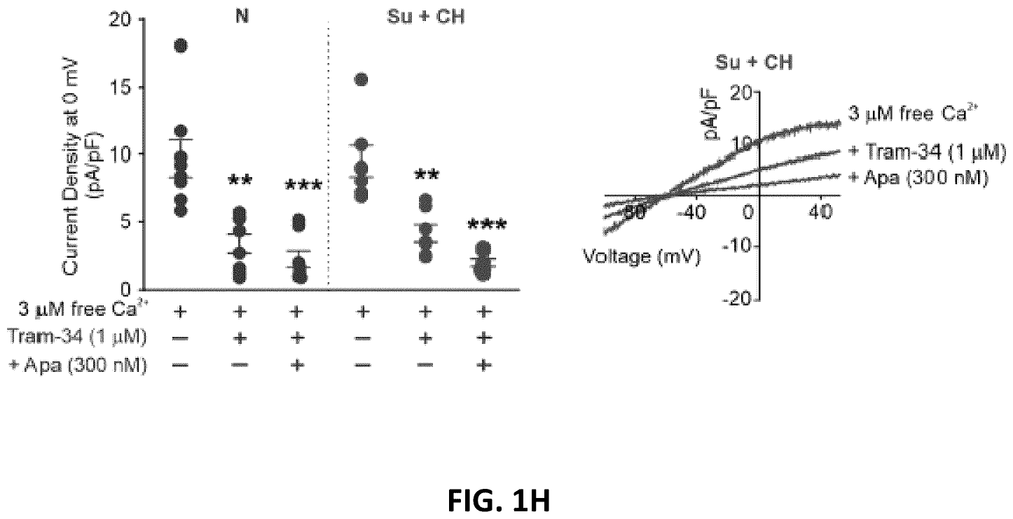

eNOS activation and prostacyclin release are impaired in PH (5-8, 18, 28-30). However, direct studies of endothelial IK/SK channel activity in PH have not been performed. The possibility that ionic currents through IK and SK channels are impaired in PH was tested. To this end, mice were used that were exposed to chronic hypoxia (CH; 4 weeks; 10% O₂) together with the vascular endothelial growth factor (VEGF) receptor antagonist SU5416 (20 mg·kg−1; s.c.; once a week), which is known to cause a more profound PH phenotype than CH alone (18, 20). Mice exposed to SU5416 plus CH (Su+CH) showed elevated right ventricular systolic pressure (RVSP), an indirect in vivo indicator of PAP, compared with normoxic (N) mice (FIG. 1A). The Fulton index, a ratio of right ventricular (RV) weight to left ventricle and septal (LV+S) weight, was also higher in mice exposed to Su+CH, confirming the development of right ventricle hypertrophy in these mice (FIG. 1B). Previous studies have established that endothelial Ca²⁺ influx, eNOS activity, and eNOS-mediated vasodilation are impaired in this model of PH (18).

Whole-cell patch-clamp experiments were performed in freshly isolated ECs from small, 4th order PAs (˜50 μm; FIGS. 1C and 1D). IK/SK channel currents induced by the IK/SK channel agonist NS309 (1 μmol/L) were recorded following application of a 250-ms voltage ramp from −100 mV to +50 mV. Outward currents at 0 mV were compared following sequential addition of 1 μmol/L NS309, 1 μmol/L TRAM-34 (IK channel inhibitor), and 300 nmol/L apamin (Apa; SK channel inhibitor; FIG. 1E). Outward currents in the presence of NS309, NS309+TRAM-34, or NS309+TRAM-34+Apa were not different between ECs from N and Su+CH groups (FIG. 1F), supporting the idea that endothelial IK and SK channel activity is not altered in this model of PH. Additionally, IK and SK channel currents in the presence of 3 μmol/L free cytosolic Ca²⁺ were not different between ECs from N and Su+CH groups, indicating that the Ca²⁺-sensitivity of IK and SK channels is not altered in this model of PH (FIGS. 1G and 1H).

Endothelial IK/SK channel-mediated dilation is unaltered in PAs from PH mice.

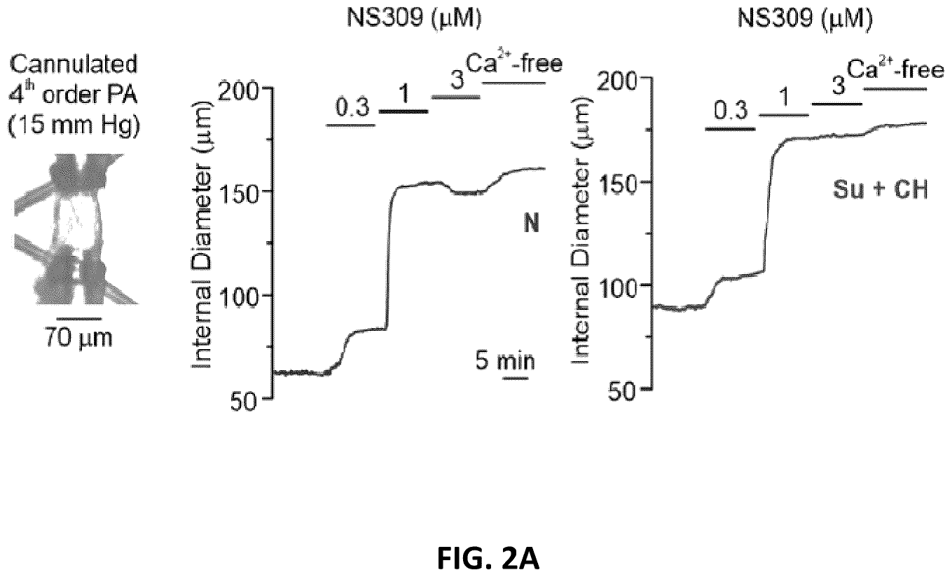

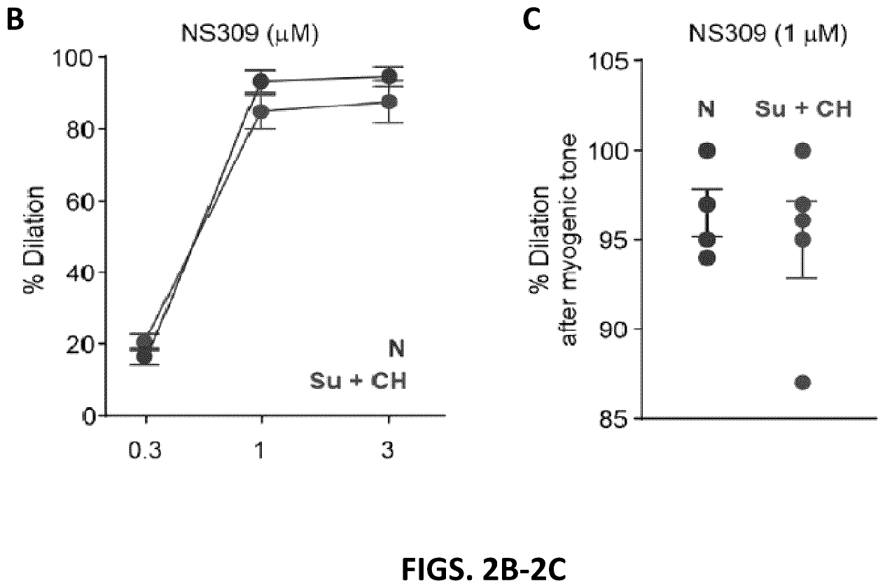

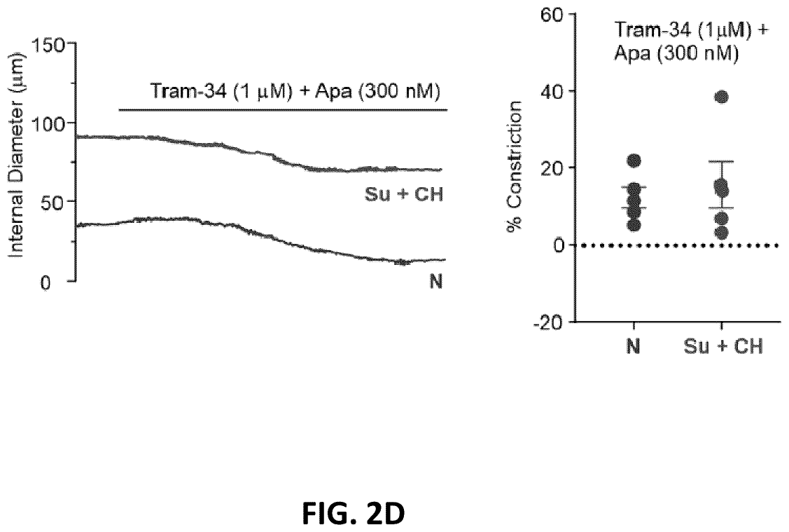

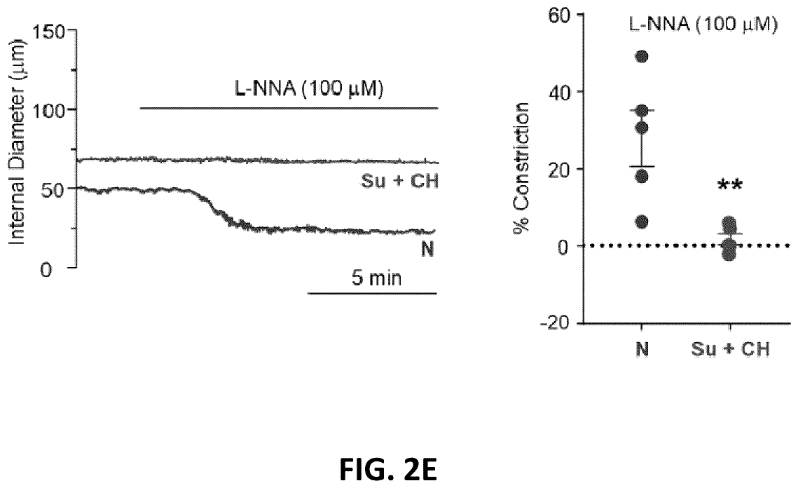

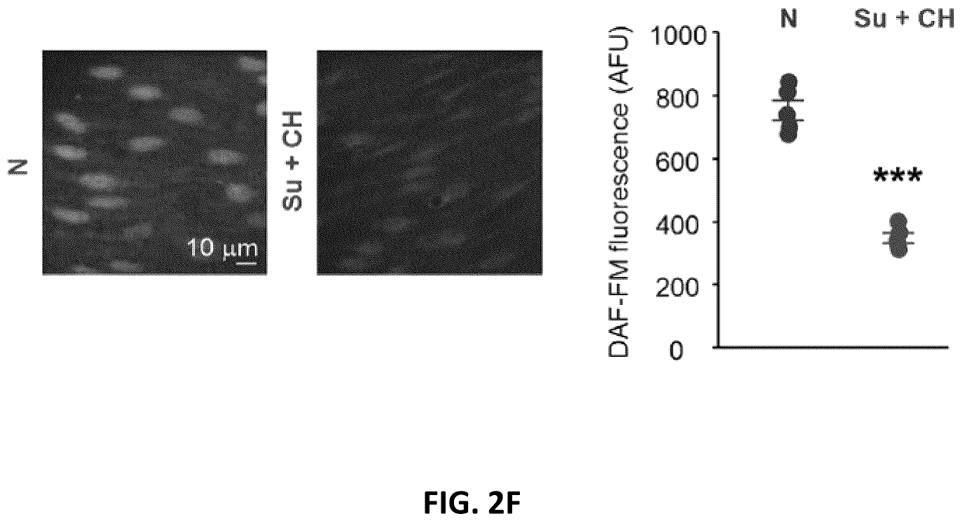

The unaltered activity of endothelial IK and SK channels in freshly isolated ECs from small PAs of Su+CH mice suggested that IK/SK channel-mediated dilation would also be unaltered in PH. To confirm this, endothelium-dependent dilation of small PAs was recorded in response to the IK/SK channel agonist NS309 (0.3-1 μmol/L) using pressure myography (17, 18) and it was found that NS309-induced dilation of small PAs was not different between N and Su+CH mice (FIGS. 2A and 2B). Similarly, in PAs that developed myogenic tone, NS309 (1 μmol/L)-induced dilation was not different between N and Su+CH groups (FIG. 2C). Given that endothelial IK/SK channel activation is known to cause dilation via electrical communication from ECs to SMC through myoendothelial gap junctions (31, 32), the absence of a change in NS309-induced vasodilation also provides evidence supporting unaltered electrical communication between ECs and SMC in PH.

The dilator effect of basal (absence of agonist) IK/SK channel was compared to the eNOS activity in small PAs from N and Su+CH mice. Exposure to TRAM-34 (1 μmol/L) and Apa (300 nmol/L) constricted small PAs by ˜10%, a constriction that was not different between N and Su+CH mice (FIG. 2D), indicating that the dilatory effect of basal IK/SK channel activity is not altered in PH. NOS inhibition with L-NNA (100 μmol/L) constricted PAs from N mice by ˜20%, but was unable constrict PAs from Su+CH mice, demonstrating the dilatory effect of basal eNOS activity in PAs from N mice and its absence in PAs from Su+CH mice (FIG. 2E). Endothelial NO levels in PAs, measured using the fluorescent NO indicator 4-amino-5 methylamino-2′,7′ difluorofluorescenin diacetate (DAF-FM; 5 μmol/L), were lower in Su+CH mice compared with N mice (FIG. 2F). Endothelial NO levels in response to purinergic receptor and transient receptor potential vanilloid 4 (TRPV4) channel agonists, which increase NO levels in ECs, were also lower in PAs from SU+CH mice (18). Together, these data establish that eNOS-mediated dilation is impaired in PH, but endothelial IK/SK channel-mediated dilation remains intact.

Acute treatment with IK/SK channel activator decreases PAP in mice exposed to Su+CH.

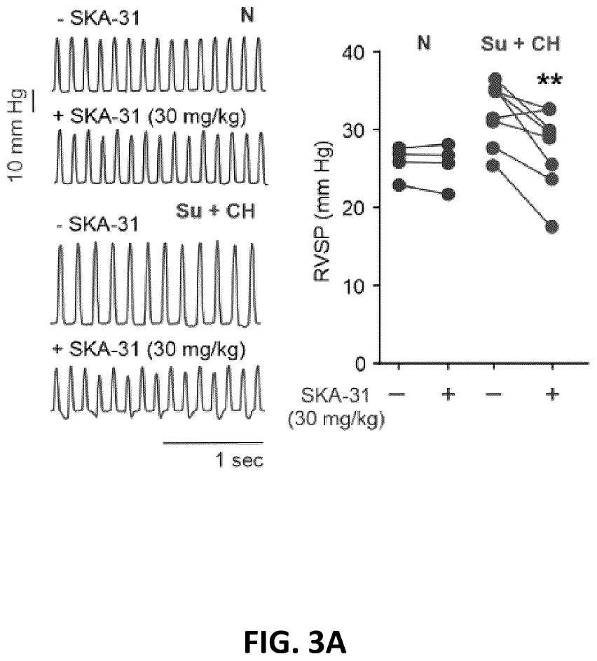

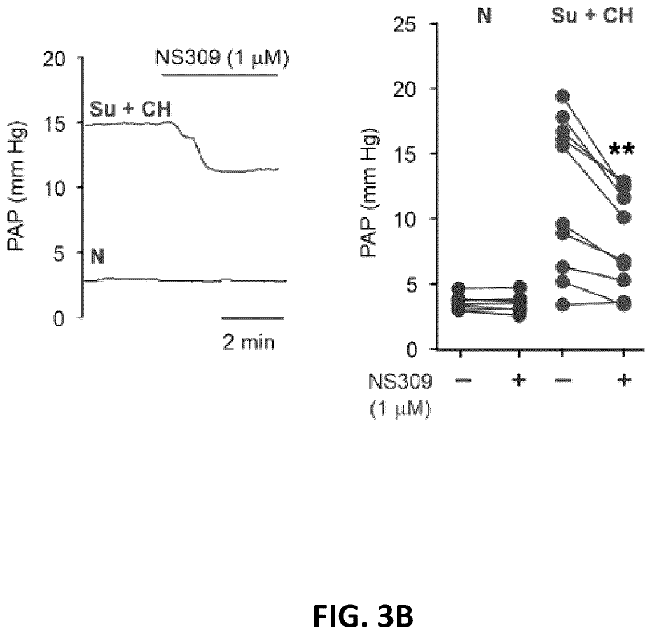

Unaltered endothelial IK/SK channel-mediated dilation of small PAs in PH led to the belief that IK/SK channel agonists would decrease PAP in PH. For in vivo studies, SKA-31 was used to activate IK/SK channels instead of NS309 (used for activating IK/SK channels in ex vivo studies) because of its favorable metabolism (33). SKA-31 administration (30 mg·kg⁻¹; i.p.) significantly reduced RVSP in Su+CH mice but not in N mice (FIG. 3A). PAP, measured in an isolated perfused lung preparation as described previously (18), was decreased by activation of IK/SK channels with NS309 (1 μmol/L) in Su+CH mice, but not in N mice (FIG. 3B). These results provide the first proof-of-principle evidence that activation of endothelial IK/SK channels can be used as a strategy to reduce PAP in PH. Moreover, these findings identify an endothelial mechanism that is not altered in PH and can be activated to lower PAP in PH.

SMCs from PAs do not Express Functional IK/SK Channels Under Normal Conditions or in PH

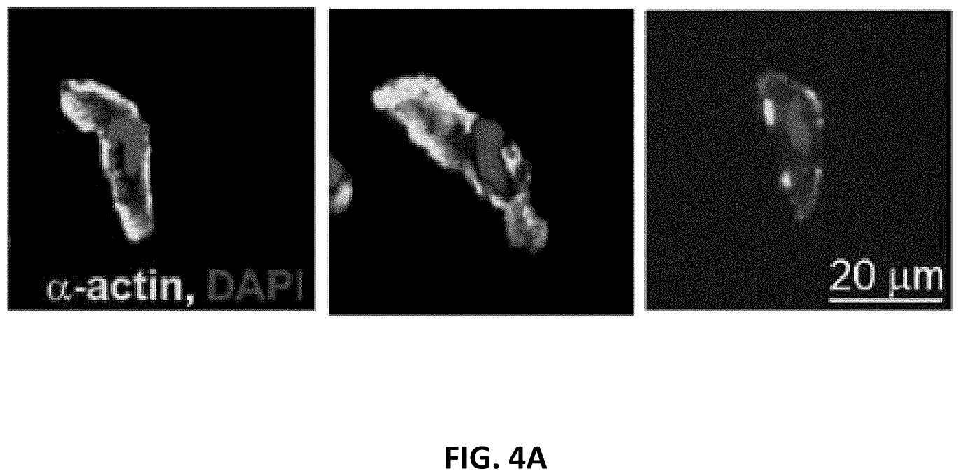

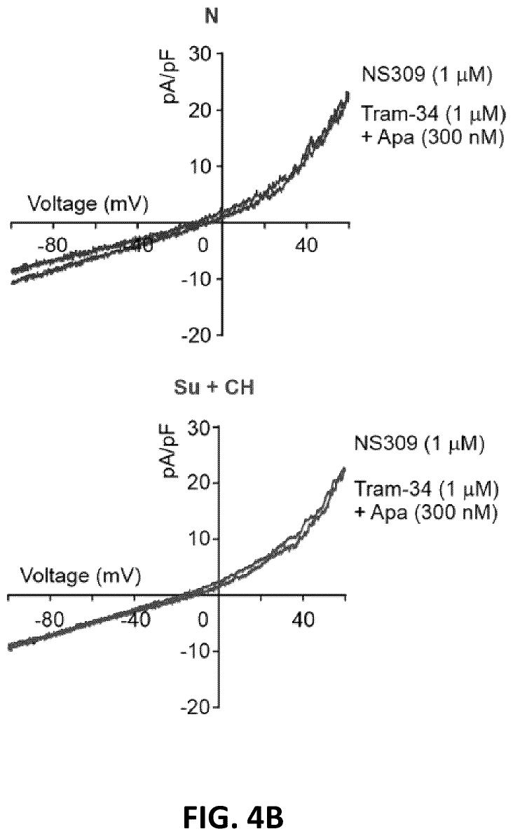

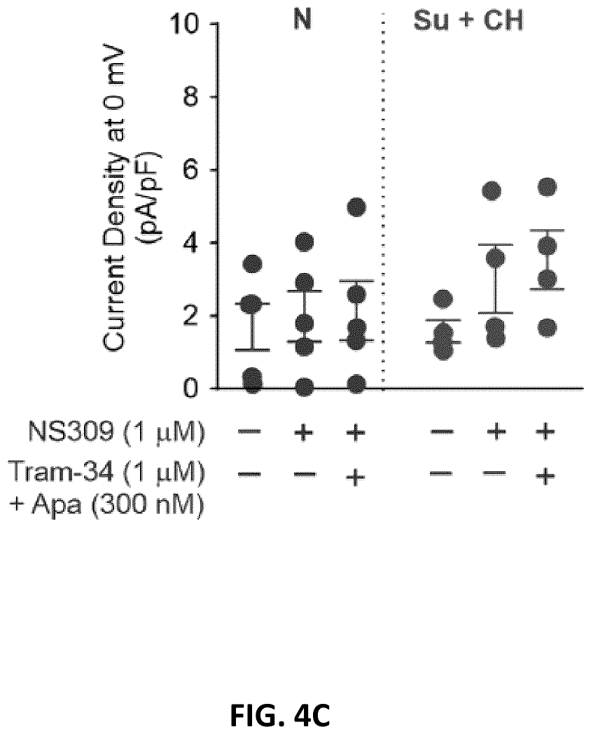

To rule out a possible role for SMC IK/SK channels in SKA-31/NS309-induced lowering of PAP, it was tested whether SMCs from small PAs exhibit IK/SK channel currents. NS309-induced ionic currents were measured in freshly isolated SMCs (FIG. 4A) from PAs in whole-cell patch-clamp experiments (FIGS. 4B and 4C). TRAM-34 (1 μmol/L) and Apa (300 nmol/L) did not reduce NS309 (1 μmol/L)-induced outward currents in SMCs from PAs of control or Su+CH mice. Moreover, outward currents at 0 m V in the presence of NS309 or NS309+TRAM-34+Apa were not different between SMCs from PAs of N and Su+CH mice. These data confirm the absence of functional IK/SK channels in SMCs from PAs under normal conditions and in PH, and support the idea that SMCs do not contribute to the dilatory or PAP-lowering effects of IK/SK channel activators in PH.

Chronic IK/SK channel activation decreases PA remodeling in a mouse model of PH.

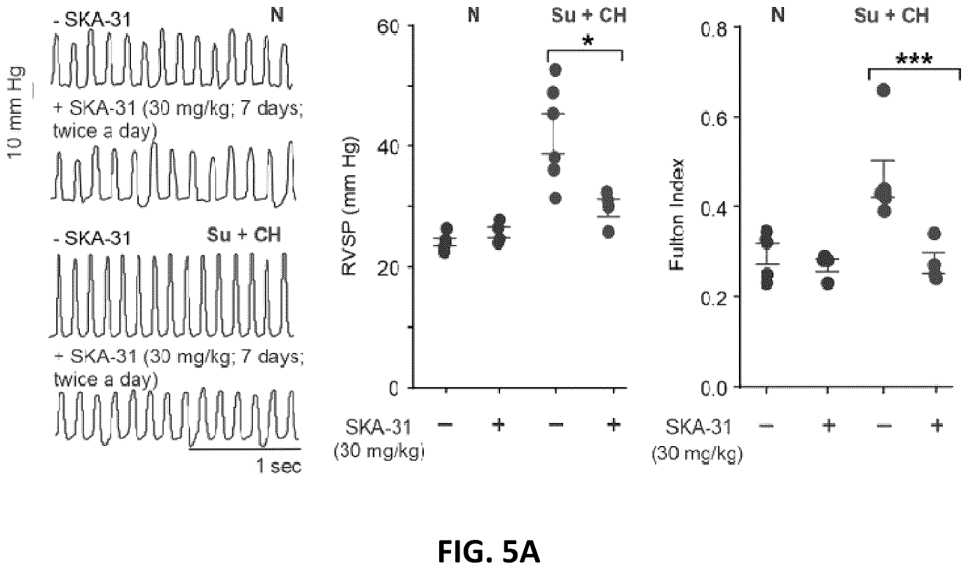

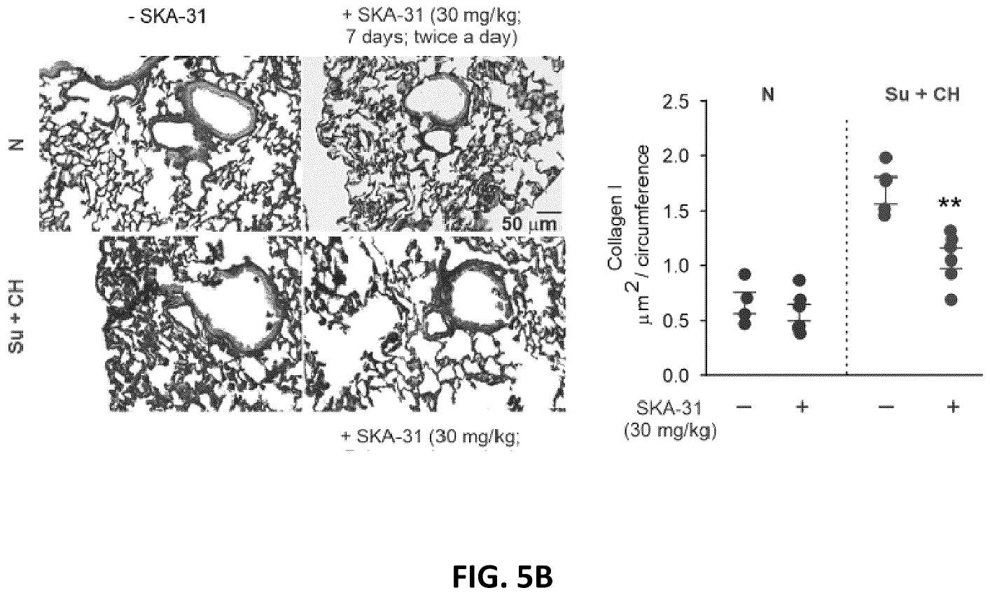

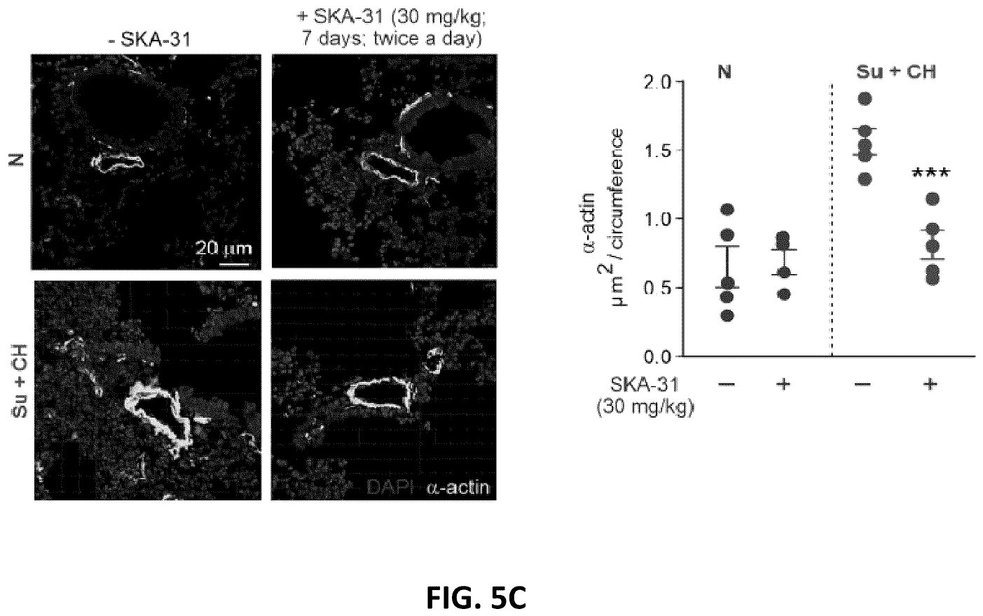

Pulmonary arterial remodeling is a hallmark of PH. Based on the finding that acute treatment with IK/SK channel activators reduces PAP in a mouse model of PH, we was believed that chronic treatment with an IK/SK channel activator would reduce PA remodeling in PH. Su+CH mice were treated with SKA-31 (30 mg·kg⁻¹; i.p.) twice a day for 7 days during the fourth week of exposure to Su+CH. Chronic SKA-31 treatment significantly lowered RVSP and the Fulton index in Su+CH mice (FIG. 5A), demonstrating beneficial effects on PAP and right ventricular hypertrophy. A morphological analysis using Masson's Trichrome staining (FIG. 5B) for collagen I and labeling of muscle fibers by immunostaining for α-actin (FIG. 5C) revealed enhanced remodeling of small PAs in Su+CH mice, a characteristic PH phenotype. Chronic treatment with SKA-31 reduced collagen content and α-actin immunostaining in Su+CH mice. These data provide the first evidence that chronic IK/SK channel activation has beneficial effects on PA remodeling in PH.

### DISCUSSION

Under physiological conditions, endothelium-dependent dilation of PAs occurs through spatially localized release of diffusible mediators from ECs (NO or prostacyclin) that act on nearby SMCs (18, 34-36). The release of NO or prostacyclin from ECs is severely impaired in PH (5-8, 18, 28-30). Therefore, currently used vasodilators lower PAP by directly elevating the levels of NO, prostacyclin, or their downstream mediators. However, elevated extracellular NO or prostacyclin can act on other cell types and have undesirable side effects. One potential strategy for limiting the side-effects of vasodilator therapy is to activate an endothelial vasodilator pathway that is not altered in PH. The studies provided herein used direct measurements of IK/SK channel currents in ECs from small PAs to provide evidence that endothelial IK/SK channel activity is not altered in the Su+CH mouse model of PH. The preserved functionality of IK/SK channels is also reflected in unaltered IK/SK channel-dependent dilation of PAs in PH. Moreover, pharmacological activation of IK/SK channels reduced RVSP and PAP in this model, and a week-long treatment with an IK/SK channel activator reduced pulmonary arterial remodeling and right ventricular hypertrophy-hallmarks of PH. Taken together, the findings indicate that pulmonary endothelial IK/SK channels represent therapeutic targets that, because they are unaltered in PH, can be activated to reduce PAP and decrease PA remodeling.

Endothelial IK/SK channel currents have not been studied in PH. There was previously provided evidence for functional IK/SK channel currents in freshly isolated ECs from small PAs and that showed activation of endothelial IK/SK channels dilates small PAs (24). Although the loss of endothelium-dependent vasodilation is well-established in PH, this effect has mostly been attributed to impaired eNOS and prostacyclin signaling. Provided herein is the first direct evidence that, unlike eNOS, endothelial IK/SK channel activity is not impaired in PH. Pharmacological activation of IK/SK channels reduces RVSP and PAP in Su+CH mice but not in normal mice, indicating that IK/SK channel activation does not reduce PAP to below physiological levels. Lack of an effect of SKA-31 on RVSP in normal mice could be explained by (1) “high-flow”-induced PA dilation, which helps maintain a low resistance in normal mice, and addition of a dilator is unable to cause further dilation; and/or (2) lower potency of SKA-31 compared to NS309 in activating IK/SK channels (37, 38). In direct contrast to its effects on resting PAP, IK/SK channel activation with SKA-31 lowered resting systemic blood pressure (33), possibly pointing to differences in basal resistance between pulmonary (low-resistance, high flow) and systemic (high resistance, low flow) vasculatures. In a clinical setting, PH is often accompanied by increased systemic blood pressure. Although the effect of IK/SK channel activation on PAP in PH was not previously known, IK/SK channel agonists have been shown to lower systemic blood pressure in systemic hypertension (37). Together with these previous findings, the data show that IK/SK channel activation in PH has additional beneficial effects on systemic blood pressure for PH patients with systemic hypertension.

Two different IK/SK channel activators were used for in vivo RVSP versus ex vivo PAP measurements. While NS309 is a highly potent activator (EC₅₀ for IK and SK channels is ˜20 nmol/L and ˜600 nmol/L, respectively), it also has a short half-life in vivo (38). SKA-31, although a less potent activator of IK/SK channels than NS309, has a longer half-life. Therefore, SKA-31 was used for in vivo studies (37). Previous studies in the literature have also used NS309 for ex vivo studies (9, 24) and SKA-31 for in vivo studies (39, 40). The PAP measurements provided herein with NS309 and RVSP measurements with SKA-31 confirmed that two different IK/SK channel activators have desirable effects on PAP and RVSP in the Su+CH mouse model of PH.

The endogenous mechanisms responsible for activation of endothelial IK/SK channels in PAs have not been investigated. eNOS and IK/SK channels appear to be the predominant mediators of endothelium-dependent dilation in systemic and pulmonary arteries. Both eNOS and IK/SK channels can be activated by increases in intracellular Ca²⁺. Multiple Ca²⁺ signals that activate IK/SK channels in systemic arteries have been identified (24, 26, 41-47). In PAs, Ca²⁺ influx through endothelial transient receptor potential vanilloid 4 (TRPV4) channels (48) activates eNOS but not IK/SK channels (24). This selective activation of eNOS by TRPV4 channels in PAs is attributable to the spatial proximity of TRPV4 channels with eNOS but not IK/SK channels. Although the activity of IK/SK channels is similar between systemic and pulmonary arteries, the Ca²⁺ signals that activate endothelial IK/SK channels in PAs are unknown. The new findings demonstrate that IK/SK channels have a dilatory effect on PAs under basal conditions, and that this dilatory effect is not altered in PH. Therefore, the discovery of endogenous mechanisms that activate endothelial IK/SK channels in PAs could unveil additional therapeutic targets for lowering PAP in PH through endothelium-dependent dilation.

Previous studies on the functional effects of endothelial IK/SK channels in PH have focused on large PAs. Simonsen and colleagues showed that exposure to chronic hypoxia decreases the dilatory response of large PAs to the IK/SK activator, NS4591, in rats (49). In another study, the dilation of large PAs (˜560 μm internal diameter) in response to the IK/SK channel agonist, NS309, was shown to increase by ˜3-fold in mice exposed to chronic hypoxia (50). The differences between the instant findings on endothelial IK/SK channel-dependent dilation of PAs in PH and previously published findings could be explained by the size of the PAs (small vs. large) used in the respective studies. Indeed, it was recently shown that the mechanisms underlying endothelium-dependent dilation are different in large (>200 μm) and small (<100 μm) PAs (10, 18, 21). It should be noted that the regulation of PAP occurs at the level of small PAs (˜50-100 μm). Therefore, studies of IK/SK channel-dependent dilation in small PAs provide more relevant information on the effect of endothelial IK/SK channel activity on PAP. Additionally, the patch-clamp studies provide the first direct recordings of endothelial IK/SK channel activity in PH and demonstrate that PH does not affect endothelial IK/SK channel activity in small PAs.

Remodeling of small PAs is a crucial contributor to elevated PAP in PH. The histological studies provide evidence that remodeling of PAs in Su+CH mice is reduced by chronic treatment with the IK/SK channel agonist, SKA-31. The precise mechanism responsible for the decrease in PA remodeling by IK/SK channel activation is not clear. Previously published data suggest that K⁺ channels in SMCs can regulate SMC proliferation in the systemic vasculature (51). However, the findings confirm that functional IK/SK channels are not present in SMCs from PAs. Therefore, it is postulated that the effect of SKA-31 on SMC proliferation and PA remodeling is a consequence of the decrease in PAP following the administration of SKA-31. IK channels have been shown to inhibit proliferation of human airway SMCs (52), T-lymphocytes (53), and fibroblasts (54). In contrast, other studies have suggested that IK channel overexpression can lead to the proliferation of systemic vascular SMCs (55). Similarly, global SK channel knock-in mice show increased proliferation and angiogenesis (41, 56), and IK/SK channel inhibitors alleviate vascular remodeling in the systemic vasculature (57, 58). Thus, the effects of IK/SK channel activity on SMC proliferation and arterial remodeling appear to be divergent in systemic and pulmonary vasculatures. These findings are consistent with the well-established structural and functional differences between systemic and pulmonary circulations.

In conclusion, this study provides compelling evidence that IK/SK channel activity in small PAs is not altered in a mouse model of PH. Activation of IK/SK channels dilates PAs and reduces PAP in PH. IK/SK channel activation also has beneficial effects on PA and cardiac remodeling, which are hallmarks of PH. These findings identify an endogenous signaling mechanism that is not altered in PH and can be targeted to decrease PAP and vascular remodeling in PH, establishing that IK/SK channel activators with desirable pharmacokinetic and pharmacodynamic properties could be used therapeutically to lower PAP and reduce vascular remodeling in PH. Identifying endogenous activators of endothelial IK/SK channels in PAs can present additional therapeutic targets for the treatment of PH.

### Example 2

IK/SK channel activation dilates small pulmonary arteries from a human subject with pulmonary hypertension (PH)

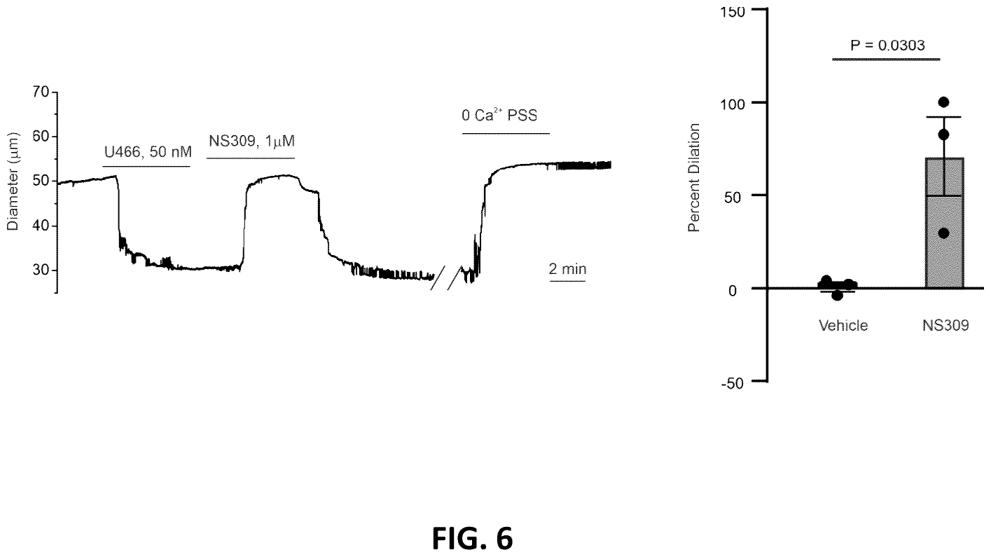

We tested whether IK/SK channel activation dilates pulmonary arteries from human subjects with PH. Deidentified human lung wedge samples were obtained from explanted lung during lung transplantation surgeries in a PH patient, in accordance with the policies of the University of Virginia Institutional Review Board (IRB #19044). Informed consent was obtained after the nature and possible consequences of the studies were explained. Multiple small pulmonary arteries (˜50 μm) were dissected and used for pressure myography experiments. Pulmonary arteries were cannulated at both ends and pressurized to a physiological intraluminal pressure of 15 mm Hg. The arteries were pre-constricted using U46619 (U466, thromboxane A2 receptor agonist, FIG. 6). Addition of NS309 (IK/SK channel agonist) dilated the pulmonary arteries from PH patient (FIG. 6). Vehicle treatment did not alter the diameter of pulmonary arteries. These results provide the first evidence that IK/SK channel agonist dilates pulmonary arteries in clinical pulmonary hypertension, further supporting the concept that IK/SK channel activation can be used as a strategy for lowering pulmonary arterial pressure in PH.

### REFERENCES CITED

1. R. L. Benza et al., An evaluation of long-term survival from time of diagnosis in pulmonary arterial hypertension from the REVEAL Registry. Chest 142, 448-456 (2012).
2. N. Galie et al., 2015 ESC/ERS Guidelines for the diagnosis and treatment of pulmonary hypertension: The Joint Task Force for the Diagnosis and Treatment of Pulmonary Hypertension of the European Society of Cardiology (ESC) and the European Respiratory Society (ERS): Endorsed by: Association for European Paediatric and Congenital Cardiology (AEPC), International Society for Heart and Lung Transplantation (ISHLT). Eur Respir J 46, 903-975 (2015).
3. K. Fukuda et al., Guidelines for the Treatment of Pulmonary Hypertension (JCS 2017/JPCPHS 2017). Circulation journal: official journal of the Japanese Circulation Society 83, 842-945 (2019).
4. R. Budhiraja, R. M. Tuder, P. M. Hassoun, Endothelial dysfunction in pulmonary hypertension. Circulation 109, 159-165 (2004).
5. J. R. Klinger, S. H. Abman, M. T. Gladwin, Nitric oxide deficiency and endothelial dysfunction in pulmonary arterial hypertension. Am J Respir Crit Care Med 188, 639-646 (2013).
6. A. Giaid, D. Saleh, Reduced expression of endothelial nitric oxide synthase in the lungs of patients with pulmonary hypertension. N Engl J Med 333, 214-221 (1995).
7. B. W. Christman et al., An imbalance between the excretion of thromboxane and prostacyclin metabolites in pulmonary hypertension. The New England journal of medicine 327, 70-75 (1992).
8. A. Giaid et al., Expression of endothelin-1 in the lungs of patients with pulmonary hypertension. The New England journal of medicine 328, 1732-1739 (1993).
9. Z. Zhang et al., Coronary endothelial dysfunction prevented by small-conductance calcium-activated potassium channel activator in mice and patients with diabetes. J Thorac Cardiovasc Surg 160, e263-e280 (2020).
10. M. Ottolini et al., Mechanisms underlying selective coupling of endothelial Ca(2+) signals with eNOS vs. IK/SK channels in systemic and pulmonary arteries. The Journal of physiology 10.1113/JP279570 (2020).
11. S. K. Sonkusare et al., AKAP150-dependent cooperative TRPV4 channel gating is central to endothelium-dependent vasodilation and is disrupted in hypertension. Science signaling 7, ra66 (2014).
12. F. R. Giachini et al., Upregulation of intermediate calcium-activated potassium channels counterbalance the impaired endothelium-dependent vasodilation in stroke-prone spontaneously hypertensive rats. Transl Res 154, 183-193 (2009).
13. Y. Liu et al., Inactivation of Endothelial Small/Intermediate Conductance of Calcium-Activated Potassium Channels Contributes to Coronary Arteriolar Dysfunction in Diabetic Patients. J Am Heart Assoc 4, e002062 (2015).
14. D. Liu et al., Increased transient receptor potential canonical type 3 channels in vasculature from hypertensive rats. Hypertension 53, 70-76 (2009).
15. R. E. Haddock et al., Diet-induced obesity impairs endothelium-derived hyperpolarization via altered potassium channel signaling mechanisms. PloS one 6, e16423 (2011).
16. M. Ottolini et al., Local Peroxynitrite Impairs Endothelial TRPV4 Channels and Elevates Blood Pressure in Obesity. Circulation 10.1161/CIRCULATIONAHA.119.043385 (2020).
17. C. Marziano et al., Nitric Oxide-Dependent Feedback Loop Regulates Transient Receptor Potential Vanilloid 4 (TRPV4) Channel Cooperativity and Endothelial Function in Small Pulmonary Arteries. J Am Heart Assoc 6 (2017).
18. Z. Daneva et al., Caveolar peroxynitrite formation impairs endothelial TRPV4 channels and elevates pulmonary arterial pressure in pulmonary hypertension. Proc Natl Acad Sci USA 118 (2021).
19. S. L. Archer et al., Nitric oxide and cGMP cause vasorelaxation by activation of a charybdotoxin-sensitive K channel by cGMP-dependent protein kinase. Proc Natl Acad Sci USA 91, 7583-7587 (1994).
20. S. H. Vitali et al., The Sugen 5416/hypoxia mouse model of pulmonary hypertension revisited: long-term follow-up. Pulm Circ 4, 619-629 (2014).
21. Z. Daneva et al., Endothelial pannexin 1-TRPV4 channel signaling lowers pulmonary arterial pressure in mice. Elife 10 (2021).
22. F. Anvari et al., Tissue-derived proinflammatory effect of adenosine A2B receptor in lung ischemia-reperfusion injury. J Thorac Cardiovasc Surg 140, 871-877 (2010).
23. A. K. Sharma et al., Pannexin-1 channels on endothelial cells mediate vascular inflammation during lung ischemia-reperfusion injury. Am J Physiol Lung Cell Mol Physiol 315, L301-L312 (2018).
24. M. Ottolini et al., Mechanisms underlying selective coupling of endothelial Ca(2+) signals with eNOS vs. IK/SK channels in systemic and pulmonary arteries. J Physiol 598, 3577-3596 (2020).
25. C. F. Wenceslau et al., Guidelines for the measurement of vascular function and structure in isolated arteries and veins. Am J Physiol Heart Circ Physiol 321, H₇₇-H₁₁₁ (2021).
26. S. K. Sonkusare et al., Elementary Ca²⁺ signals through endothelial TRPV4 channels regulate vascular function. Science 336, 597-601 (2012).
27. Y. L. Chen et al., Novel Smooth Muscle Ca(2+)-Signaling Nanodomains in Blood Pressure Regulation. Circulation 146, 548-564 (2022).
28. C. Xue, R. A. Johns, Endothelial nitric oxide synthase in the lungs of patients with pulmonary hypertension. The New England journal of medicine 333, 1642-1644 (1995).
29. V. V. Mclaughlin, M. D. McGoon, Pulmonary arterial hypertension. Circulation 114, 1417-1431 (2006).
30. R. M. Tuder et al., Prostacyclin synthase expression is decreased in lungs from patients with severe pulmonary hypertension. Am J Respir Crit Care Med 159, 1925-1932 (1999).
31. G. Edwards, M. Feletou, A. H. Weston, Endothelium-derived hyperpolarising factors and associated pathways: a synopsis. Pflugers Arch 459, 863-879 (2010).
32. Y. Yamamoto, M. F. Klemm, F. R. Edwards, H. Suzuki, Intercellular electrical communication among smooth muscle and endothelial cells in guinea-pig mesenteric arterioles. J Physiol 535, 181-195 (2001).
33. M. Damkjaer et al., Pharmacological activation of KCa3.1/KCa2.3 channels produces endothelial hyperpolarization and lowers blood pressure in conscious dogs. Br J Pharmacol 165, 223-234 (2012).
34. J. G. Gerber, N. Voelkel, A. S. Nies, I. F. McMurtry, J. T. Reeves, Moderation of hypoxic vasoconstriction by infused arachidonic acid: role of PGI2. J Appl Physiol Respir Environ Exerc Physiol 49, 107-112 (1980).
35. S. Hara et al., Overexpression of prostacyclin synthase inhibits growth of vascular smooth muscle cells. Biochemical and biophysical research communications 216, 862-867 (1995).
36. M. W. Geraci et al., Pulmonary prostacyclin synthase overexpression in transgenic mice protects against development of hypoxic pulmonary hypertension. J Clin Invest 103, 1509-1515 (1999).
37. A. Sankaranarayanan et al., Naphtho[1,2-d]thiazol-2-ylamine (SKA-31), a new activator of KCa2 and KCa3.1 potassium channels, potentiates the endothelium-derived hyperpolarizing factor response and lowers blood pressure. Mol Pharmacol 75, 281-295 (2009).
38. D. Strobaek et al., Activation of human IK and SK Ca²⁺-activated K+channels by NS309 (6,7-dichloro-1H-indole-2,3-dione 3-oxime). Biochim Biophys Acta 1665, 1-5 (2004).
39. C. M. John et al., SKA-31, an activator of Ca(2+)-activated K(+) channels, improves cardiovascular function in aging. Pharmacol Res 151, 104539 (2020).
40. J. Radtke, K. Schmidt, H. Wulff, R. Kohler, C. de Wit, Activation of KCa3.1 by SKA-31 induces arteriolar dilatation and lowers blood pressure in normo- and hypertensive connexin40-deficient mice. Br J Pharmacol 170, 293-303 (2013).
41. M. S. Taylor et al., Altered expression of small-conductance Ca²⁺-activated K+(SK3) channels modulates arterial tone and blood pressure. Circ Res 93, 124-131 (2003).
42. J. Ledoux, A. D. Bonev, M. T. Nelson, Ca²⁺-activated K+channels in murine endothelial cells: block by intracellular calcium and magnesium. J Gen Physiol 131, 125-135 (2008).
43. S. Earley, A. L. Gonzales, R. Crnich, Endothelium-dependent cerebral artery dilation mediated by TRPA1 and Ca²⁺-Activated K+channels. Circ Res 104, 987-994 (2009).
44. P. Bagher et al., Low intravascular pressure activates endothelial cell TRPV4 channels, local Ca²⁺ events, and IKCa channels, reducing arteriolar tone. Proc Natl Acad Sci USA 109, 18174-18179 (2012).
45. C. H. Tran et al., Endothelial Ca²⁺ wavelets and the induction of myoendothelial feedback. Am J Physiol Cell Physiol 302, C1226-1242 (2012).
46. P. W. Pires, M. N. Sullivan, H. A. Pritchard, J. J. Robinson, S. Earley, Unitary TRPV3 channel Ca²⁺ influx events elicit endothelium-dependent dilation of cerebral parenchymal arterioles. Am J Physiol Heart Circ Physiol 309, H2031-2041 (2015).
47. M. N. Sullivan et al., Localized TRPA1 channel Ca²⁺ signals stimulated by reactive oxygen species promote cerebral artery dilation. Science signaling 8, ra2 (2015).
48. Y. L. Chen, S. K. Sonkusare, Endothelial TRPV4 channels and vasodilator reactivity. Curr Top Membr 85, 89-117 (2020).
49. C. Kroigaard et al., K(Ca)3.1 channel downregulation and impaired endothelium-derived hyperpolarization-type relaxation in pulmonary arteries from chronically hypoxic rats. Experimental physiology 98, 957-969 (2013).
50. C. Wandall-Frostholm et al., Pulmonary hypertension in wild type mice and animals with genetic deficit in KCa2.3 and KCa3.1 channels. PLOS One 9, e97687 (2014).
51. C. B. Neylon, R. J. Lang, Y. Fu, A. Bobik, P. H. Reinhart, Molecular cloning and characterization of the intermediate-conductance Ca(2+)-activated K(+) channel in vascular smooth muscle: relationship between K(Ca) channel diversity and smooth muscle cell function. Circ Res 85, e33-43 (1999).
52. M. C. Shepherd et al., KCa3.1 Ca²⁺ activated K+channels regulate human airway smooth muscle proliferation. Am J Respir Cell Mol Biol 37, 525-531 (2007).
53. H. Wulff et al., Design of a potent and selective inhibitor of the intermediate-conductance Ca²⁺-activated K+channel, IKCal: a potential immunosuppressant. Proc Natl Acad Sci USA 97, 8151-8156 (2000).
54. S. G. Rane, A Ca²⁺-activated K+current in ras-transformed fibroblasts is absent from nontransformed cells. The American journal of physiology 260, C104-112 (1991).
55. R. Kohler et al., Blockade of the intermediate-conductance calcium-activated potassium channel as a new therapeutic strategy for restenosis. Circulation 108, 1119-1125 (2003).
56. C. C. Rada et al., Overexpression of the SK3 channel alters vascular remodeling during pregnancy, leading to fetal demise. Am J Physiol Endocrinol Metab 303, E825-831 (2012).
57. I. Grgic et al., Selective blockade of the intermediate-conductance Ca²⁺-activated K+channel suppresses proliferation of microvascular and macrovascular endothelial cells and angiogenesis in vivo. Arterioscler Thromb Vasc Biol 25, 704-709 (2005).
58. K. Toyama et al., The intermediate-conductance calcium-activated potassium channel KCa3.1 contributes to atherogenesis in mice and humans. J Clin Invest 118, 3025-3037 (2008).

We claim:

1. A method for treating pulmonary hypertension (PH) in a subject in need thereof, the method comprising administering to the subject a therapeutically effective amount of an activator of at least one of an endothelial intermediate potassium (IK) channel and an endothelial small potassium (SK) channel.

2. The method of claim 1, wherein the activator activates both of the IK channel and the SK channel.

3. The method of claim 1, wherein the activator is administered as a chronic treatment.

4. The method of claim 3, wherein the activator is administered over the course of at least 1 week.

5. The method of claim 4, wherein the activator is administered at least once per day for at least 7 days.

6. The method of claim 4, wherein the activator is administered at least twice per day for at least 7 days.

7. The method of claim 1, wherein the activator is a compound.

8. The method of claim 7, wherein the activator comprises at least one of SKA-31, NS309, and NS4591 or a pharmaceutically acceptable salt thereof.

9. The method of claim 7, wherein the activator is SKA-31 or a pharmaceutically acceptable salt thereof.

10. The method of claim 7, wherein the activator is NS309 or a pharmaceutically acceptable salt thereof.

11. The method of claim 7, wherein the activator is NS4591 or a pharmaceutically acceptable salt thereof.

12. The method of claim 1, wherein the subject is a human.
